(19) United States
(12) Patent Application Publication
(10) Pub. No.: US 2026/0194864 A1
(43) Pub. Date: Jul. 9, 2026

(54) ELECTRONIC DEVICE

(71) Applicant: Apple Inc., Cupertino, CA (US)

(72) Inventors: Junlin Mu, Redwood City, CA (US); Adam T. Clavelle, San Francisco, CA (US); Antonio F. Herrera, Woodside, CA (US); Brad G. Boozer, Campbell, CA (US); Daniela M. Deschamps, Palo Alto, CA (US); Devon K. Copeland, San Francisco, CA (US); Eric T. Chiang, San Francisco, CA (US); Ryan Sellden, Encinitas, CA (US); Sameer Pandya, Sunnyvale, CA (US); Tyler S. Bushnell, San Francisco, CA (US); Ian M. Fisher, Mountain View, CA (US)

(21) Appl. No.: 19/320,183

(22) Filed: Sep. 5, 2025

Related U.S. Application Data
(60) Provisional application No. 63/742,635, filed on Jan. 7, 2025.

(51) Int. Cl. G04G 17/04; G04G 17/06; H01Q 1/27; H05K 5/00; H05K 5/02

(52) U.S. Cl. CPC G04G 17/04; G04G 17/06; H01Q 1/273; H05K 5/0086; H05K 5/0217

(57) ABSTRACT

An electronic device includes a display assembly, a sidewall defining an internal volume including four corners, an elongate conductor extending between the display assembly and the sidewall the elongate conductor including a plurality of contact points spaced around a perimeter of the display assembly, each contact point of the plurality of contact points being welded to the housing, the plurality of contact points including a discrete welded contact point in each of the four corners. The electronic device can also include a printed circuit board (PCB) disposed in the internal volume, and an electronical connector contacting the PCB. The electrical connector can contact the elongate member and forming an electrical contact between the sidewall and the PCB.

### CROSS-REFERENCE TO RELATED APPLICATIONS

This application claims the benefit of U.S. Provisional Application No. 63/742,635, filed 7 Jan. 2025, entitled “ELECTRONIC DEVICE,” the entire disclosure of which is hereby incorporated by reference.

### FIELD

The present disclosure relates generally to electronic devices. More particularly, the present disclosure relates to wearable electronic devices.

### BACKGROUND

Electronic devices are increasingly being designed with device portability in mind, for example, to allow users to use these devices in a wide variety of situations and environments. In the context of wearable devices, these devices can be designed to include many different functionalities and to be operated in many different locations and environments. The components of an electronic device, for example, the processors, memory, antennas, display, and other components can partially determine a level of performance of the electronic device. Further, the arrangement of these components with respect to one another in the device can also determine the level of overall performance of the electronic device.

Continued advances in electronic devices and their components have enabled considerable increases in performance. Existing components and structures for electronic devices can, however, limit the levels of performance of such devices. For example, while some components can achieve high levels of performance in some situations, the inclusion of multiple components in devices sized to enhance portability can limit the performance of the components, and thus, the performance of the device. Consequently, further tailoring and arrangement of components for electronic devices to provide additional or enhanced functionality, without introducing or increasing undesirable device properties, can be desirable.

### SUMMARY

In at least one example of the present disclosure, an electronic device can include a display assembly, a sidewall defining an internal volume including four corners, an elongate conductor extending between the display assembly and the sidewall the elongate conductor including a plurality of contact points spaced around a perimeter of the display assembly, each contact point of the plurality of contact points being welded to the housing, the plurality of contact points including a discrete welded contact point in each of the four corners. The electronic device can also include a printed circuit board (PCB) disposed in the internal volume, and an electronical connector contacting the PCB. The electrical connector can contact the elongate member and forming an electrical contact between the sidewall and the PCB.

In some examples, the sidewall can include an upper portion, a lower portion, and a non-conductive split disposed between the upper portion and the lower portion. Additionally, the electronic device can further include an antenna assembly including a resonator, the resonator including the upper portion. In some example, the PCB includes an antenna ground contact, and the electrical connector contacts the antenna ground contact. The electrical connector can be welded to the elongate conductor, in some examples.

In some examples of the electronic device, there can be twelve discrete contact points, and each contact point can be a discrete contact point. In some examples, the elongate conductor further includes an elongate body, wherein each contact point is independently rotated from the elongate body.

In some embodiments, an electronic device can include a conductive housing sidewall defining an internal volume, a printed circuit board (PCB) disposed in the internal volume, an electrical connector contacting the PCB, and an elongate conductive member disposed between the housing sidewall and the electrical connector. In some examples, the elongate conductive member contacts the electrical connector and is welded to housing sidewall. In some examples, the elongate conductive member defines a plurality of contact points, wherein each contact point of the plurality of contact points is decoupled and rotated from the elongate body and welded to the housing.

In some examples, the elongate conductive member contacts the electrical connector at a first location along a length of the elongate conductive member. In some examples, the elongate conductive member contacts the electrical connector at a third location along the length of the elongate conductive member, and the elongate conductive member is welded to the housing sidewall at a fourth location along the length of the elongate conductive member. In some examples, the conductive housing sidewall can be a resonating element of an antenna. In some examples the elongate conductive member can be welded to the resonating element. According to some examples, the electrical connector is a continuous component disposed adjacent the PCB. In some examples, the electrical connector extends through an insulating material between the PCB and the conductive housing sidewall. In other examples the conductive housing defines an opening, the electronic device further includes a display disposed in the opening, and the elongate conductive member is disposed between the display and the conductive housing.

In at least one embodiment, a wearable electronic device includes a housing sidewall including a lower portion and an electrically conductive upper portion separated from the lower portion by a non-conductive material, the housing sidewall defining an opening, a display component disposed in the opening, an antenna ground contact, and a display printed circuit board (display PCB) electrically coupled to the antenna ground contact. According to this example, a wave ring can be disposed between the electrically conductive upper portion and the display component, the wave ring welded to the electrically conductive upper portion at a plurality of discrete contact points disposed along a length of the wave ring, and a connector can form an electrical pathway between the wave ring and the antenna ground contact. In this example, the wearable electronic device can further include a sensor PCB disposed within the housing sidewall and a monopole antenna disposed on the sensor PCB that unifies the electrically conductive upper portion with the sensor PCB.

In some examples, the upper conductive portion and the sensor PCB each forms a resonating element of the monopole configuration. In some examples, the monopole antenna includes a coax connection to the sensor PCB. In some examples, the monopole antenna includes a four-layer flex.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A is a perspective view of an example of a wearable electronic device;

FIG. 1B shows a top perspective view of a portion of the wearable electronic device;

FIG. 1C shows a bottom perspective view of a portion of the wearable electronic device;

FIG. 2 shows an exploded perspective view of an example of a wearable electronic device;

FIG. 3A shows an exploded perspective view of an example of a wearable electronic device;

FIG. 3B shows a top perspective view of an example of a sidewall housing of an example of a wearable electronic device;

FIG. 4A shows a cross-sectional side view of a sidewall and internal components of an example of a wearable electronic device;

FIG. 4B shows another cross-sectional side view of a sidewall and internal components of an example of a wearable electronic device;

FIG. 5A shows a top view of a subassembly of a wearable electronic device;

FIG. 5B shows a top view of a portion of an example of a wearable electronic device including a printed circuit board (PCB) and surrounding electrical contacts;

FIG. 6A shows a top view of one or more grounds of a wearable electronic device;

FIG. 6B shows a cross-sectional view of a ground of a wearable electronic device;

FIG. 7A shows a top view of a monopole antenna ground of a wearable electronic device;

FIG. 7B shows a cross-sectional side view of a monopole antenna ground of a wearable electronic device;

FIG. 7C shows a top perspective view of a monopole antenna ground of a wearable electronic device;

FIG. 8A shows a top view of a monopole antenna ground of a wearable electronic device;

FIG. 8B shows a cross-sectional view of a monopole antenna ground of a wearable electronic device;

FIG. 8C shows a top perspective view of a monopole antenna ground of a wearable electronic device;

FIG. 9 shows a cross-sectional view of a wearable electronic device;

FIG. 10A shows a close-up cross-sectional view of a wearable electronic device;

FIG. 10B shows a side view and a close-up cross sectional view of a wearable electronic device;

FIG. 10C shows a side cross-sectional view of a subsystem of a wearable electronic device;

FIG. 10D-10F show various views of an antennae ground ring of a wearable electronic device;

FIG. 11 shows a cross-sectional view of a portion of an electronic device;

FIG. 12 shows another cross-sectional view of a portion of an electronic device;

FIG. 13 shows a top cross-sectional view of an electronic device;

FIG. 14 shows another cross-sectional view of a portion of an electronic device;

FIG. 15 shows another cross-sectional view of a portion of an electronic device;

FIG. 16 shows another cross-sectional view of a portion of an electronic device;

FIG. 17 shows a cross-sectional view of a portion of an example of an electronic device;

FIG. 18 shows a cross-sectional view of a portion of an example of an electronic device;

FIG. 19 shows a front perspective view of a portion of an example of an electronic device;

FIG. 20A shows a top view of a battery cell of a wearable electronic device;

FIG. 20B shows an exploded view of a battery cell of a wearable electronic device;

FIG. 20C shows a cross-sectional view of a battery cell of a wearable electronic device;

FIG. 21 shows a top view of a battery cell within a wearable electronic device;

FIG. 22A shows a perspective view of a crown assembly for an electronic device;

FIG. 22B shows a perspective view of a portion of a crown assembly for an electronic device;

FIG. 22C shows a cross-sectional view of a portion of a crown assembly for an electronic device;

FIG. 22D shows a position sensor of a crown assembly for an electronic device; and

FIG. 23 shows an example transparent cover for an electronic device.

### DETAILED DESCRIPTION

Reference will now be made in detail to representative examples illustrated in the accompanying drawings. It should be understood that the following descriptions are not intended to limit the embodiments to one preferred example or embodiment. To the contrary, it is intended to cover alternatives, modifications, and equivalents as can be included within the spirit and scope of the described embodiments as defined by the appended claims.

The following disclosure generally relates to electronic devices. More particularly, the present disclosure relates to wearable electronic devices. The wearable electronic devices of the present disclosure include tailored arrangements of components to provide additional or enhanced functionality, without introducing or increasing undesirable device properties or performance. In this way, more functionality and componentry can be included in wearable devices for users to wear and operate in any condition or activity without limiting the functionality and durability of the devices.

In some examples, a wearable electronic device includes a welded wave ring that is selectively and discretely welded to portions of the upper housing of the device to ensure a secure electrical connection that enhances antenna performance, particularly in wet conditions. In some examples the wave ring is welded at each of the four corners of the upper housing to mitigate signal loss in wet conditions. In other examples, the antenna configuration is a monopole configuration that unifies an electrically conductive upper portion of the device housing, which acts as a radiating element of the antenna, with the system PCB. The incorporation of a monopole antenna configuration provides a significant increase in antenna performance, relative to traditional wearable antenna configurations.

In some additional examples, a flexible yet planar ground ring with a continuous conductive surface is provided to ensure consistent and reliable electrical connection, while preventing undesired forces that can effect adjacent components and displays.

In some examples an efficient environmental sensor and microphone combined sensor configuration is used including a shared housing port that simplifies sealing configurations, part count, and allows for secure sealing and predictable liquid ejection paths. Additionally, a rigid perforated plate covers the microphone and environmental sensor to reduce reverberations between the combined volumes.

Additional efficiencies and configuration are provided, including a stacked battery configuration that includes a top and bottom plate welded to a thicker sidewall. This configuration allows for a compact form factor, eliminates the need for a space-occupying lip or ridge, allows for cutouts and unique non-square and non-linear battery shapes to maximize power capacity while fitting in unique space geometries, and allow for brackets and components to be welded to the battery housing for efficient mounting and placement.

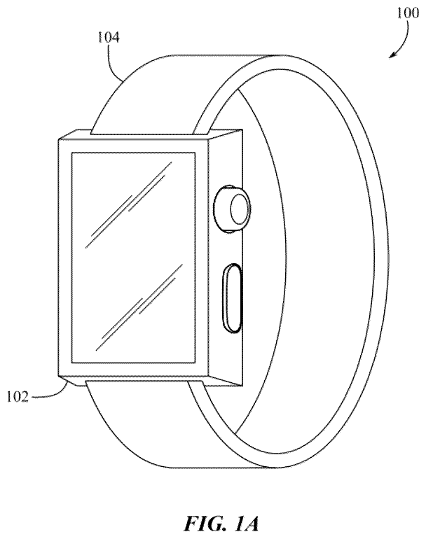

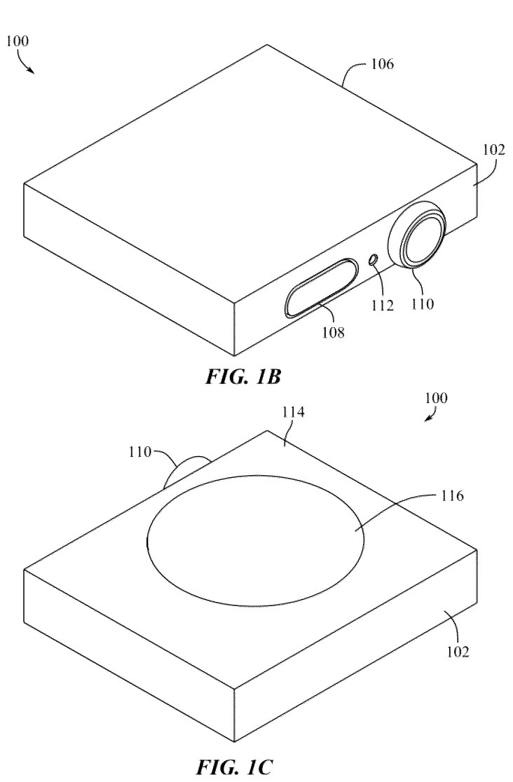

Specific examples and embodiments of electronic devices, including wearable electronic devices, exemplifying the above-mentioned benefits and configurations are discussed below with reference to FIGS. 1-23. However, those skilled in the art will readily appreciate that the detailed description given herein with respect to these Figures is for explanatory purposes only and should not be construed as limiting. Furthermore, as used herein, a system, a method, an article, a component, a feature, or a sub-feature comprising at least one of a first option, a second option, or a third option should be understood as referring to a system, a method, an article, a component, a feature, or a sub-feature that can include one of each listed option (e.g., only one of the first option, only one of the second option, or only one of the third option), multiple of a single listed option (e.g., two or more of the first option), two options simultaneously (e.g., one of the first option and one of the second option), or combination thereof (e.g., two of the first option and one of the second option).

FIG. 1A shows an example of an electronic device 100. The electronic device shown in FIG. 1A is a watch, such as a smartwatch. The smartwatch of FIG. 1A is merely one representative example of a device that can be used in conjunction with the systems and methods disclosed herein. Electronic device 100 can correspond to any form of wearable electronic device, a portable media player, a media storage device, a portable digital assistant (“PDA”), a tablet computer, a computer, a mobile communication device, a GPS unit, a remote-control device, or other electronic device. The electronic device 100 can be referred to as an electronic device, or a consumer device. In some examples, the electronic device 100 can include a housing 102 that can carry operational components, for example, in an internal volume at least partially defined by the housing. The electronic device 100 can also include a strap 104, or other retaining component that can secured the device 100 to a body of a user as desired. Further details of the electronic device are provided below with reference to FIG. 1B.

FIG. 1B illustrates the electronic device 100, for example a smartwatch, which can be substantially similar to and can include some or all of the features of the devices described herein, including the electronic device 100 shown in FIG. 1A but without the strap 104. The device 100 can include a housing 102, and a display assembly 106 attached to the housing 102. The housing 102 can substantially define at least a portion of an exterior surface of the device 100.

The display assembly 106 can include a glass, a plastic, or any other substantially transparent exterior layer, material, component, or assembly. The display assembly 106 can include multiple layers, with each layer providing a unique function, as described herein. Accordingly, the display assembly 106 can be, or can be a part of, an interface component. The display assembly 106 can define a front exterior surface of the device 100 and, as described herein, this exterior surface can be considered an interface surface. In some examples, the interface surface defined by display assembly 106 can receive inputs, such as touch inputs, from a user.

In some examples, the housing 102 can be a substantially continuous or unitary component and can define one or more openings to receive components of the electronic device 100. In one example, the substantially continuous or unitary component is not formed of multiple components joined together. The continuous component or member can be formed as a single, unitary piece without seams, connections, or multiple parts. In some examples, the device 100 can include input components such as one or more buttons 108 and/or a crown 110 that can be disposed in the openings. In some examples, a material can be disposed between the buttons 108 and/or crown 110 and the housing 102 to provide an airtight and/or watertight seal at the locations of the openings. The housing 102 can also define one or more openings or apertures, such as aperture 112 that can allow for sound to pass into or out of the internal volume defined by the housing 102. For example, the aperture 112 can be in communication with a microphone component disposed in the internal volume. In some examples, the housing 102 can define or include a feature, such as an indentation to removably couple the housing 102 and a strap or retaining component.

FIG. 1C shows a bottom perspective view of the electronic device 100. The device 100 can include a back cover 114 that can be attached to the housing 102, for example, opposite the display assembly 106. The back cover 114 can include ceramic, plastic, metal, or combinations thereof. In some examples, the back cover 114 can include an at least partially electromagnetically transparent component 116. The electromagnetically transparent component 116 can be transparent to any desired wavelengths of electromagnetic radiation, such as visible light, infrared light, radio waves, or combinations thereof. In some examples, the electromagnetically transparent component 116 can allow sensors and/or emitters disposed in the housing 102 to communicate with the external environment. Together, the housing 102, display assembly 106 and back cover 114 can substantially define an internal volume and an external surface of the device 100.

Any of the features, components, and/or parts, including the arrangements and configurations thereof shown in FIGS. 1A-1C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 1A-1C.

As noted above, portable and wearable electronic devices can be designed to be used in many different environments and during any kind of activity throughout a user's day. For example, wearable electronic watches, headphones, and phones can be carried by a user during exercise, sleep, driving, biking, hiking, swimming, diving, outside in the rain, outside in the sun, and so forth. Wearable electronic devices described herein are configured to withstand the varied and often harsh conditions of various environments, including changing environments and wet environments. Wet environments can include wearing devices in the rain or when submerged during bating or swimming, for example.

Examples of electronic devices disclosed herein include components, features, arrangements, and configurations that resists damage and corrosion due to exposure to moisture. Some aspects of devices described herein can include gaps between components through which moisture, water, or other fluids could enter. The gaps may be present for aesthetic purposes or for functional purposes. However, one or more components, including epoxy seals, insulating materials and frames, and other components of devices described herein can be configured to prevent such moisture from entering into the internal volume of the device where sensitive electronic component could be damaged thereby.

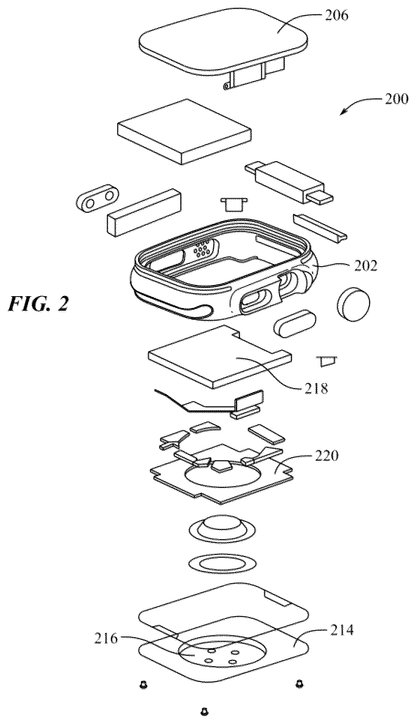

Along these lines, FIG. 2 illustrates an exploded view of another example of an electronic device 200, which can also be a portion of a wearable electronic watch or other wearable electronic device. The exploded assembly 200 may show how various structural and functional elements are organized in a vertical stack configuration to form the complete device structure. Device 200 includes a display assembly 206, housing 202, back cover 214, and electromagnetically transparent component 216. In addition, the exploded view of FIG. 2A illustrates various internal components that may be disposed within an internal volume defined by the housing 202, back cover 214, electromagnetically transparent component 216, and display assembly 206. For example, the device 200 can include one or more printed circuit boards (PCBs) 218 and one or more antenna components 220, electrical connectors and flexes, antenna ground rings, buttons, seals, gaskets, memory components, processors, sensors, dials, batteries, and so forth.

Any of the features, components, and/or parts, including the arrangements and configurations thereof shown in FIG. 2 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 2.

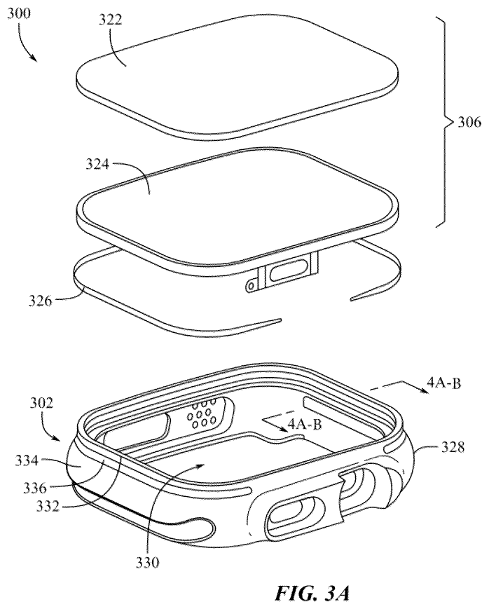

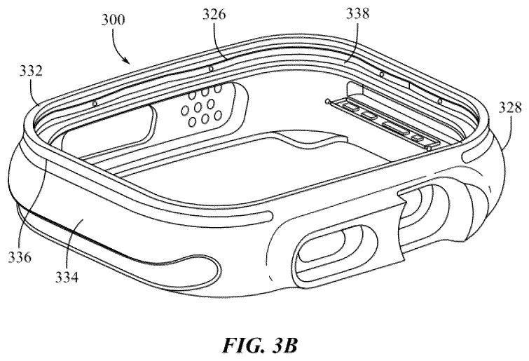

FIG. 3A illustrates a close-up view of a portion of the exploded view of the device 300 shown in FIG. 2 (device 200), including the housing 302 and the display assembly 306, with the display assembly further exploded to illustrate the display cover 322 and display layers 324. In addition, the exploded view of FIG. 3A shows a wave ring 326 (also referred to herein as an “elongate conductive member”), which will be described and discussed in more detail hereafter with reference to other figures. In at least one example, the housing 302 includes sidewall or sidewalls 328 that define an internal volume and an opening 330. When assembled, the display assembly 306 or one or more components of the display assembly 306 can be disposed in the opening to form an outer surface of the device 300 and define the internal volume. The housing 302 can include an oleophobic coating. For example, one or more portions of the housing 302, such as the sidewall 328, or components thereof, can include an oleophobic coating.

In at least one example, the sidewall 328 can include an upper portion 332 and a lower portion 334. The upper portion 332 and the lower portion 334 can be separated by a middle portion 336 disposed between the upper portion 332 and the lower portion 334. In at least one example, the upper portion 332 and the lower portion 334 of the sidewall 328 can include one or more electrically conductive materials and the middle portion 336 can include one or more electrically non-conductive materials and/or an insulating material. The middle portion 336 can be molded to or otherwise adhered to the upper portion 332 and/or the lower portion 334 such that the upper portion 332, the lower portion 334, and the middle portion 336 form a single, unitary sidewall 328 of the housing 302, as shown.

Along these lines, FIG. 3B shows a top perspective view of a subassembly of a device 300, according to the present disclosure. The subassembly includes housing sidewall 328 that includes the upper portion 332, lower portion 334, and middle portion 336 separating the upper portion 332 from the lower portion 334. As noted above, the upper portion 332 and the lower portion 332 can include electrically conductive material and the middle portion 336 can include electrically insulating or non-conductive material such that the upper portion 332 of the sidewall 328 forms a resonating element of an antenna separated by a distance in the vertical or “Z” direction (or a “Z-distance”) relative to the electrical grounding plane of the lower portion 334. An epoxy component 338 can also be bonded to an inside of the sidewall 328 and to the middle portion 636 and the lower portion 634. In addition, the wave ring 326 is also shown in FIG. 3B.

Any of the features, components, and/or parts, including the arrangements and configurations thereof shown in FIGS. 3A-3B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 3A-3B.

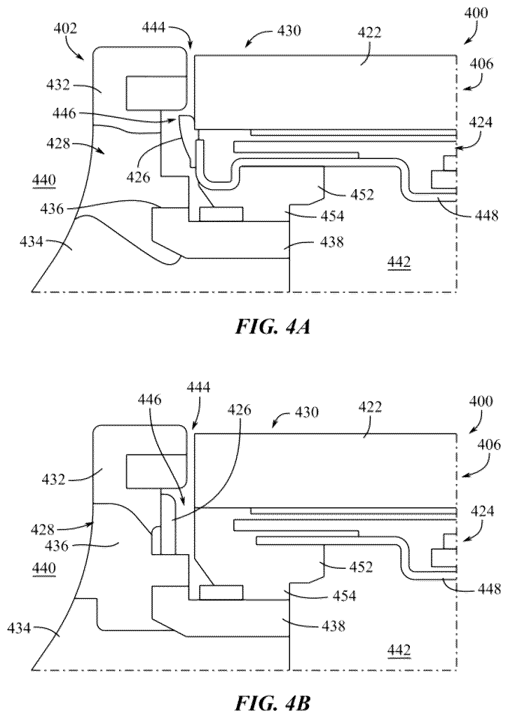

FIGS. 4A-4B illustrate a cross-sectional view of a device 400 with a display assembly 406 disposed in the opening 432 formed by the sidewall 428. In at least one example, the display assembly 406, which includes the display cover 422 and one or more other display layers 424 disposed below the display cover 422, can be disposed in the opening 430 such that a gap 444 is formed between the display assembly 406 and the sidewall 428. The gap 444 can be understood as a space between the display assembly 406, or the display cover 422 thereof, and the sidewall 428, or upper portion 432 thereof, wherein the display cover 422 does not contact the sidewall 428. In at least one example, an upper surface of the display cover 422 can be flush with, or disposed lower than, an upper surface of the upper portion 432.

In at least one example, a cavity 446 is formed in which the wave ring 426 is disposed. The cavity 446 can be defined by the sidewall 428, including the upper portion 432 and the middle portion 436, the epoxy component 438, and the display assembly 406 or at least the display cover 422 thereof. In at least one example, the cavity 446 can also be defined by an insulating material 424 disposed between the display assembly 406 and/or display cover 422 thereof and the epoxy component 438. One or more other components, including an LTH layer 454 or other layers. As noted above, the epoxy component 438 can bond to other layers and components, including the LTH layer 454, middle portion 436, lower portion 434, and/or the insulating material 452 to prevent moisture from entering an internal volume 442 from an external environment 440 of the device 400, such that any moisture or fluids entering the cavity 446 through the gap 444 do not continue on into the internal volume 442. In this way, the cavity 446 can be fluid tight.

More specifically, FIGS. 4A-4B show cross-sectional views at various locations around the sidewall 428 to illustrate how the wave ring 426 disposed in the cavity 446 can contact the upper portion 432 of the sidewall 428 at one or more locations along a length of the wave ring 426, as shown in FIG. 4B, and contact an electrical contact 448 on the other side of the cavity 446 at one or more other locations along the length of the wave ring 426, as shown in FIG. 4A.

Accordingly, in at least one example of the present disclosure, the housing sidewall 428 can define an opening 430 and a display component, such as the display cover 422, can be disposed in the opening 430 to form the gap 444 between the housing sidewall 428 and the display component. In at least one example, the cavity 446 is defined by the sidewall 428 and the display cover 422 with the cavity 446 in fluid communication with the external environment 440 through the gap 444. In at least one example, the epoxy component 438 at least partially defines the cavity 446 and can be in direct contact with the housing sidewall 428. The housing sidewall 428 can include an oleophobic coating. For example, an outer portion of the housing side 428, or components thereof can include an oleophobic coating.

In at least one example of the electronic device 400, the housing sidewall 428 has an upper sidewall portion 432 and a lower sidewall portion 434 bonded to a middle sidewall portion 436 disposed between the upper and lower sidewall portions 432, 434, respectively. The housing 402 can define the opening 430 and the display assembly 406 can be disposed in the opening 430 to form the gap 444 between the housing 402 and the display assembly 406. Also, in at least one example, the epoxy component 438 can serve as a seal disposed underneath the display assembly 406 and extend laterally across the gap 444 with the epoxy component seal 438 bonded directly to the middle portion 436 of the sidewall 428.

In at least one example of the present disclosure, the electronic device 400 can include the sidewall 428 defining the internal volume 442 and the opening 430. In at least one example, the sidewall 428 can include an upper portion 432, a lower portion 434, and a middle portion 436 disposed between and bonded to the upper portion 432 and the lower portion 434. The device 400 can also include the display cover 422 disposed in the opening 432 and defining the internal volume 452, the side cavity 446 defined by the display assembly 406 and the sidewall 428, with the cavity 446 in fluid communication with an external environment 440 through the gap 444 formed between the display assembly 406 and the sidewall 428, and an epoxy layer 438 contacting the lower portion 434 and the middle portion 436, and at least partially defining the cavity 446.

The device 400 can include the epoxy component 438 at least partially disposed between the display cover 422 of the display assembly 406 and the lower portion 434, or between one or more other components of the display assembly 406, including the display layers 424, and the lower portion 434. One or more other components can also be disposed or stacked between the epoxy component 438 and the display assembly 406 or cover 422, for example the LTH layer 454. In addition, one or more examples of the device 400 can include an insulating material 452. The insulating material 452 can include and support a printed circuit board (PCB) 450 disposed in the internal volume 442.

As noted above, FIGS. 4A and 4B show cross-sectional views at various locations around the sidewall 428 to illustrate how the wave ring 426 disposed in the cavity 446 can contact the upper portion 432 of the sidewall 428 at one or more locations along a length of the wave ring 426, as shown in FIG. 4B. Accordingly, in at least one example, the middle portion 436 can include gaps or windows through which columns or other portions of the upper portion 432 of the sidewall 428 are exposed through the middle portion 436 such that the wave ring 426 can contact the upper portion 432 directly, as shown in FIG. 4B. Also, the wave ring 426 can contact an electrical contact 448 on the other side of the cavity 446 at one or more other locations along the length of the wave ring 426, as shown in FIG. 4A. The electrical contact 448 can extend through the insulating material 452 and electrically connect to the PCB 450. In this way, the upper portion 432 of the sidewall 428 can be electrically connected to the PCB 450 through the wave ring 426.

In at least one example, the upper portion 432 of the sidewall 428 can be electrically isolated from the lower portion 434 via the intermediary and non-conductive middle portion 436. In this way, the upper portion 432 can be a resonating element of an antenna of the device 400 with the lower portion 434 of the sidewall 428 acting as an electrical grounding plane relative to the resonating plane of the upper portion 432. As noted above, the upper portion 432 can be electrically connected to the PCB 450 of the device 400 such that signals received and sent by the resonating upper portion 432 can be directed to the PCB 450 and can be processed with one or more processors or other electronic components of the device 400, including any processors or other electronic components mounted on the PCB 450.

The wearable electronic devices described herein can include antennas configured to send and receive electromagnetic signals during use. Incorporating effective antennas into small, compact devices such as wearable electronic watches can be challenging because the greater the distance between a resonating plane and a grounding plane of an antenna, the better the performance of the antenna will be. However, space is often limited to create the required Z-distances necessary in compact wearable electronic devices. In devices described herein, the housing and sidewalls of the device can be electrically separated into multiple portions to create resonating elements and grounding elements of an antenna with sufficient separation (Z-distance) therebetween for the housing itself to function as an antenna. However, this design has its own challenges, including electrically connecting the resonating element to a PCB, processor, or other electronic device without reducing the Z-distance of the antenna. Wearable electronic devices described herein are configured to overcome these challenges.

Any of the features, components, and/or parts, including the arrangements and configurations thereof shown in FIGS. 4A-4B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 4A-4B.

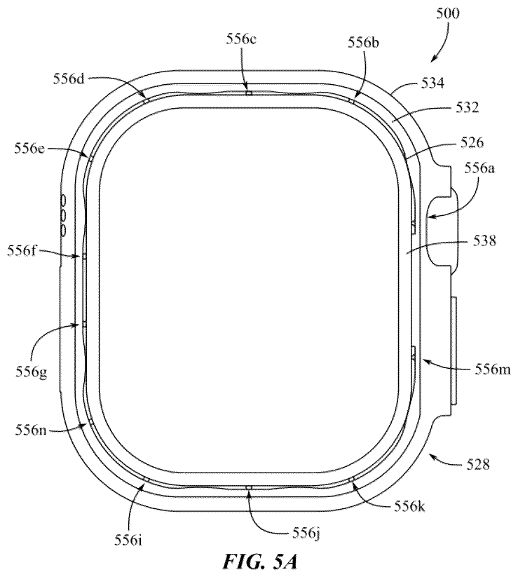

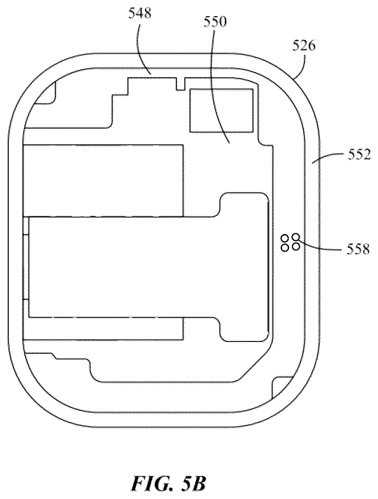

FIG. 5A shows a top view of a subassembly of a wearable electronic device 500. The subassembly includes housing sidewall 528 that includes the upper portion 532, lower portion 534, and middle portion 536 separating the upper portion 532 from the lower portion 534. As noted above, the upper portion 532 and the lower portion 532 can include electrically conductive material and the middle portion 536 can include electrically insulating or non-conductive material such that the upper portion 532 of the sidewall 528 forms a resonating element of an antenna separated by a distance in the vertical or “Z” direction (or a “Z-distance”) relative to the electrical grounding plane of the lower portion 534. The epoxy component 538 can also be bonded to an inside of the sidewall 528 and to the middle portion 536 and the lower portion 534. In addition, the wave ring 526 is also shown in FIG. 5A.

The wave ring 526 can be welded to the housing sidewall 528 at a plurality of discrete contact points 556a-556m. The plurality of contact points 556a-556m can be discrete contact points disposed along a length of the wave ring 526. One or more of the plurality of contact points 556a-556m can be positioned along a side portion of the wave ring 526. One or more of the plurality of contact points 556a-556m can be position along a corner portion of the wave ring 526. The wave ring 526 can include four to twelve of the plurality of contact points 556a-556m. In one example, the wave ring 526 can be curved back and forth in a serpentine manner around the perimeter between contact with the housing, such as the housing sidewall 528, at the plurality of contact points 556a-556m, and contact with the electrical connector. One or more of the plurality of contact points 556a-556m can be welded to the housing, such as the housing sidewall 528. In some examples, the discrete contact points 556a-556m can be formed along the perimeter of the wave ring and the portions to be welded to the housing sidewall 528 can be decoupled from the remainder of the wave ring, such that the potions to be welded to the housing can be independently rotated and deflected to facilitate the welding connection. According to this example, the remainder of the wave ring has a substantially consistent profile and direction, providing increased air gap and less inadvertent coupling.

Referring to FIG. 5B, the electrical connectivity between the wave ring 526 and internal electronic components may be established through specific interface arrangements. The wave ring 526 may be positioned to interface with internal circuitry while maintaining proper electrical isolation from other components.

A printed circuit board 550 may be positioned within the electronic device 500 to support electronic components and circuitry. The printed circuit board 550 may include conductive traces, mounting pads, and connection points for interfacing with other elements of the electronic device 500. In some cases, the printed circuit board 550 may include antenna ground contacts or other connection points for establishing electrical communication with antenna elements.

An insulating material 552 may surround the printed circuit board 550 to provide electrical isolation and structural support. The insulating material 552 may separate the printed circuit board 550 from surrounding conductive elements while allowing controlled electrical connections to pass through. In some cases, the insulating material 552 may be formed through injection molding or other processes to create precise geometries around the printed circuit board 550.

An electrical contact 548 such as an antenna ground ring may form a continuous conductive element that extends around the printed circuit board 550. The electrical contact 548 may be configured to interface with the wave ring 526 while providing electrical connectivity to the printed circuit board 550. In some cases, the electrical contact 548 may be formed as a unitary piece that maintains consistent electrical properties around the perimeter of the printed circuit board 550. According to one example, the wave ring 526 is welded to the antenna ground ring in each of the four corners of the system. In some examples, ensuring contacts between the wave ring and the antenna ground ring in the four corners improves performance of the antenna system under wet conditions. More specifically, if water enters the gap between the display and the housing during wet use-cases, ensuring secure contacts, such as welded contacts with the wave ring in the corners, the performance loss is minimized, and wet use case performance is enhanced.

The electrical contact 548 may include multiple connection areas 558 that extend onto and contact the printed circuit board 550. The connection areas 558 may provide electrical pathways between the electrical contact 548 and conductive traces or components on the printed circuit board 550. In some cases, the connection areas 558 may be positioned at specific locations to interface with antenna ground contacts or other electrical elements on the printed circuit board 550.

The arrangement of the wave ring 526, electrical contact 548, printed circuit board 550, insulating material 552, and connection areas 558 may establish electrical communication between the upper portion 532 of the housing sidewall 528 and internal electronic components. The wave ring 526 may contact the electrical contact 548 at various points along the length of the conductor while maintaining contact with the upper portion 532 through the contact points 556a through 556m. In some cases, this configuration may allow antenna signals received by the upper portion 532 to be transmitted to processing circuitry on the printed circuit board 550 through the electrical pathway established by the wave ring 526 and electrical contact 548.

Any of the features, components, and/or parts, including the arrangements and configurations thereof shown in FIGS. 5A-5B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 5A-5B.

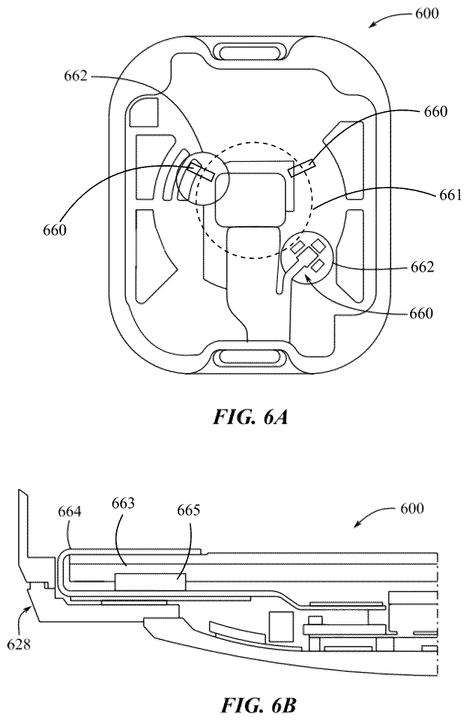

Referring to FIGS. 6A-6B, a monopole antenna 600 may be configured to provide antenna functionality within the electronic device structure. The exemplary monopole antenna 600 detailed in FIGS. 6A and 6B may demonstrate how specific structural arrangements and component interfaces can establish antenna grounding while accommodating the compact form factors of wearable electronic devices.

FIGS. 6A and 6B show a top view and a cross-sectional view of a wearable electronic device with one or more grounds 660 or contacts and pockets 662 for monopole antennas. The grounds 660 can include a pocket 662 to receive a portion of a four-layer flex 664 configured to enable a monopole antenna construction and operation. In other examples, the grounds or contacts 660 can traverse the substrate and bridge to the sensor board or sensor PCB (illustrated with by dashed line 661) below the substrate. In this way the sensor board metal is integrated into the antenna, thereby increasing the total antenna surface area. The four-layer flex 664 can extend against the antenna towards the housing sidewall 628 and wrap around components of the wearable display device 600 to establish electrical connections while accommodating geometric constraints of the device structure. The flex layer 664 may maintain electrical continuity while allowing for mechanical flexibility during assembly and operation. According to one example, the incorporation of a monopole antenna configuration provides a significant increase in antenna performance, including the ability for satellite transmission, in some examples. The monopole configuration unifies the radiating element with the sensor board or PCB becoming part of that radiating element. In some examples the sensor board or PCB is a different sensor board than the display PCB 450 discussed above, such as a rear sensor board associated with the back crystal of the wearable display device 600. According to the embodiment illustrated in FIG. 6B, the four-layer flex 664 may be substantially flat and can be biased down against the antenna to prevent desensitization or de-sensing, which is a loss of sensitivity due to noise sources. In order to prevent de-sensing, the connection of the four-layer flex 664 with the antenna can be facilitated by a more rigid and flat four-layer flex 664 and/or the inclusion of a biasing source imparting a force pressing the four-layer flex 664 against the antenna. As illustrated in FIG. 6B, a foam element 665 can be positioned between the SiP 663 and the four-layer flex 664. While illustrated as a foam element 665, any number of biasing materials or elements can be used to press the four-layer flex 664 against the antenna.

Any of the features, components, and/or parts, including the arrangements and configurations thereof shown in FIGS. 6A-6B can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 6A-6B.

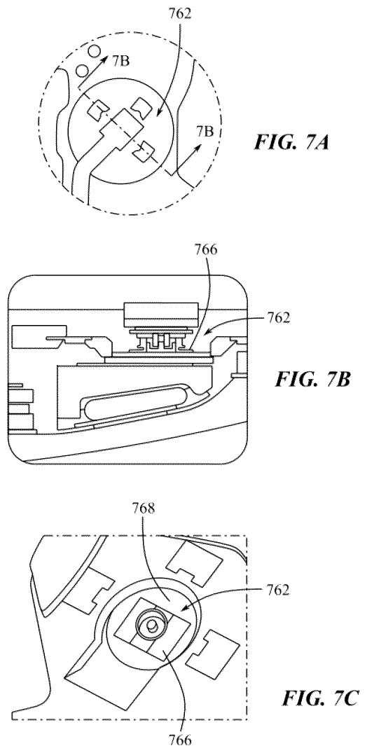

FIGS. 7A-7C show a top view, a cross sectional view, and a top perspective view of connection components of the monopole antenna of FIGS. 6A-6B. According to one example, a pocket 762 can be indented in relation to other components of the wearable display device, such as a frame (e.g., an injection molded frame). The pocket 762 can receive a sheet metal component 768 facilitating connection of a cable, such as a monopole coax cable. The pocket 762 can receive coax cable 766. The coax cable 766 can be soldered into the pocket 662 for a secure connection in a fixed position. According to some examples, the coax cable connection provides a reliable signal transmission, durability, and secure installation. Additionally, a coaxial connection can reduce signal interference and loss relative to other connection types and can provide a strong and stable connection for antennas.

Any of the features, components, and/or parts, including the arrangements and configurations thereof shown in FIGS. 7A-7C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 7A-7C.

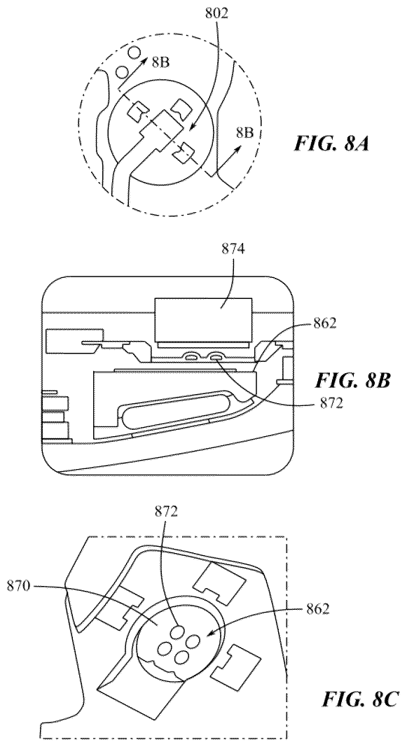

FIG. 8A-8C show a top view, a cross sectional view, and a top perspective view, respectively of another example of a connection for the monopole antenna of FIGS. 6A-6B. As illustrated in FIG. 8A-8C, the pocket 862 can be indented in relation to other components of the wearable display device, such as a frame (e.g., an injection molded frame). The pocket 862 can receive a sheet metal component 870. The sheet metal component 870 can include plating of a conductive metal (such as gold) on a portion thereof. The sheet metal component 870 can be soldered into the pocket 862. The sheet metal component 880 can include one or more dimples 872. The dimples 872 can extend toward a flex 864, such as the four-layer flex. The flex 864 can include a flat plate positioned toward the dimples 872. The flat plate can include gold plating. The flex 864 can further include a silicone piece 874. According to some examples, the silicone piece 874 can be compressed, thereby applying pressure to create a secure electrical connection between the flex 864 and the sheet metal component 870. While a compressible silicone piece 874 is illustrated and described herein, any number of biasing elements can be used to ensure a secure electrical connection between the flex 864 and the sheet metal component 870 including, but in no way limited to a spring finger. This exemplary configuration allows for a secure and predictable connection while keeping the flex 864 flat thereby allowing for some slight misalignment. These secure and reliable connections allow for the incorporation of, and improved antenna performance from, a monopole antenna system.

Any of the features, components, and/or parts, including the arrangements and configurations thereof shown in FIGS. 8A-8C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 8A-8C.

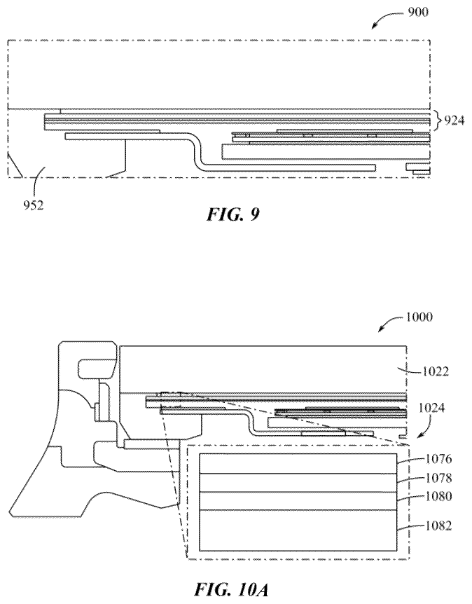

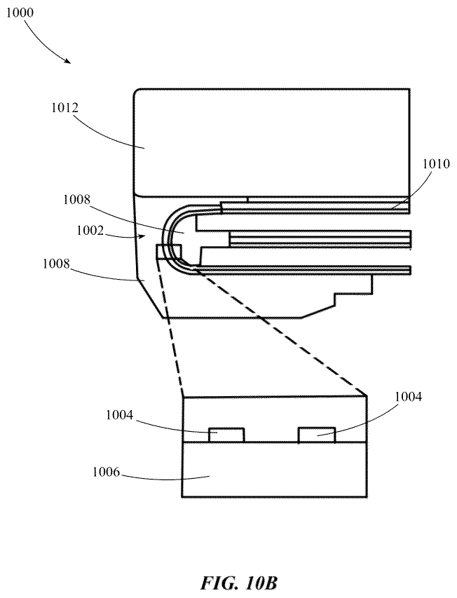

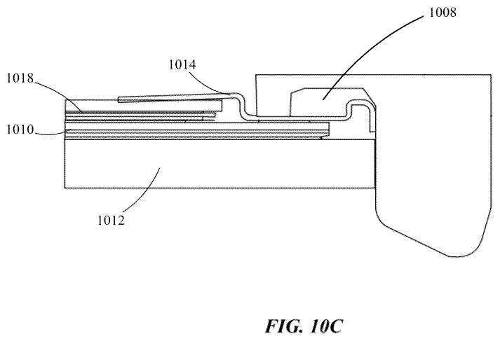

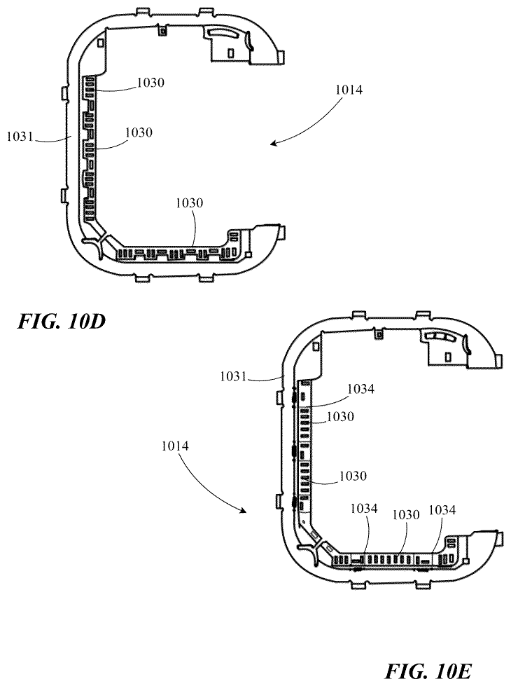

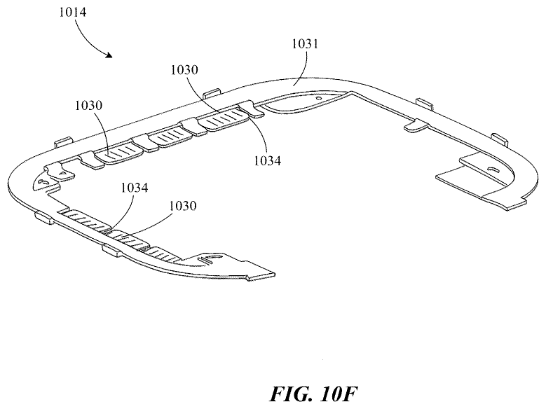

FIG. 9 shows a cross sectional view of a wearable electronic device 900. The wearable device 900 includes the insulating material 952 and display layers 924. To increase a size of the display area, a smaller boarder can be implemented on the display layers 924, within a housing of the wearable display device 900. The boarder can be an insulating material 952. The insulating material 952 can encapsulate the display layers 924. The insulating material 952 can be injected to form around the display layers 924. When injecting the insulating material 952, the display layers 924 can be subject to pressure, for example, a high pressure. The high pressure can cause waviness in the display. To combat the waviness, the display layers 924 can be configured as described in FIG. 10. In another example, the configuration of the tool used for injecting the insulating material 952 can be modified to include a rib.

Any of the features, components, and/or parts, including the arrangements and configurations thereof shown in FIG. 9 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 9.

FIG. 10A shows a close-up cross-sectional view of a wearable electronic device 1000. The wearable electronic device 1000 can include display layers 924. The display layers 1024 can include an optically clear adhesive 1076, a polarizer 1078, a display panel 1080, and a structural layer 1082.

The optically clear adhesive 1076 can be configured to adhere the display layers 1024 to a display cover 1022. The optically clear adhesive 1076 can have a thickness of about 25 microns to about 75 microns. In another example, the optically clear adhesive 1076 can have a thickness of about 40 microns to about 60 microns. In another example, the optically clear adhesive 1076 can have a thickness of about 50 microns. In some examples, a thickness of the optically clear adhesive 1076 can impact waviness. For example, a smaller thickness can reduce waviness while maintaining sufficient adhesion.

The polarizer 1078 can be an optical film that controls the amount of light in the display layers 924. The polarizer 1078 can have a thickness of about 40 microns to about 80 microns. For example, the polarizer 1078 may have a thickness of about 60 microns. The display panel 1080 can be configured to produce an image.

The structural layer 1082 can provide structure to the display layers 1024. The structural layer 1082 can have a thickness of about 75 microns to about 150 microns. In another example, the structural layer 1082 can have a thickness of about 100 microns to about 150 microns. In another example, the structural layer 1082 can have a thickness of about 125 microns.

FIG. 10B shows a cross-sectional view of a portion of an electronic device 1000. The electronic device 1000 includes multiple layers arranged in a stacked configuration. A cover glass 1012 is positioned at the top of the assembly, with a display layer 1010 disposed beneath it. As illustrated in FIG. 10B, the flex driving the display layer 1010 curves around a panel bend 1002 to orient with the display layer.

As shown, the assembly includes areas of low injection pressure overmolding 1008 positioned at multiple locations within the structure, particularly around the flex or panel bend 1002. The panel bend can include polyimide substrate 1006 extends through portions of the device 1000, with traces 1004 integrated into the structure. According to one embodiment, in order to enhance the robustness of the trades 1004 along the panel bend, the traces are formed directly on the rigid polyimide substrate that forms a part of the panel bend, rather than on a more flexible substrate. When on more flexible substrates, the traces can be susceptible to cracking due to fatigue if the bend experiences frequent or prolonged pressure variations, such as when worn by a scuba diver, sky diver, or mountain climber. Additionally, in some examples the radius of the panel bend can be increased to relieve stresses in the traces 1004. According to one example, the radius of the panel bend can range from 0.40 mm to 0.5 mm. In other examples the bend can have an internal radius of approximately 0.45 mm. The traces 1004 follow a panel bend 1002 in the configuration, allowing electrical connectivity while accommodating the curved geometry of the bend.

The cross-sectional view illustrates how these components are arranged vertically within the electronic device 1000, with each layer contributing to the overall functionality of the device. The low injection pressure overmolding 1008 appears at multiple points in the assembly, providing structural support, added sealing, and protection for other components. The panel bend 1002 demonstrates how the traces 1004 and polyimide substrate 1006 can be configured to follow curved paths within the device 1000.

FIG. 10C shows a cross-sectional view of a portion of an electronic device. The figure illustrates a stacked arrangement of components including a cover glass 1012 positioned at the top of the assembly. Below the cover glass 1012 is a display layer 1010.

A PCB 1018 is positioned beneath these display components. An antenna ground ring 1014 extends between portions of the assembly. The low injection pressure overmolding 1008 or liquid silicone rubber is integrated into the structure, providing protection for the internal components.

The cross-sectional view demonstrates how these components are arranged vertically within the device assembly. The antenna ground ring 1014 follows a curved path between the other components. The low injection pressure overmolding 1008 is positioned to encapsulate portions of the assembly while maintaining the structural integrity of the device.

The arrangement shows how the display components (cover glass 1012 and display layer 1010) are integrated with the PCB 1018 and antenna ground ring 1014 in a compact configuration. The low injection pressure overmolding 1008 provides environmental protection while allowing for proper functioning of the display and antenna components. As shown, pressures exerted by the antenna ground ring 1014 can be translated through the PCB 1018 and imprint on the display or otherwise affect the display if the antenna ground ring is not flat and/or is unduly rigid.

FIGS. 10D-10E show orthogonal views of different configurations of an antenna ground ring 1014. As shown in FIG. 10D, the antenna ground ring 1014 can include a continuous contact surface 1031 on its outer edge, providing a constant surface engagement. Additionally, a number of connection points 1030 can be formed and are disposed on the internal edge of the antenna ground ring 1014. The connection points can be spaced and positioned to correspond to connection points on a corresponding PCB (not shown). According to one embodiment, the continuous contact surface 1031 enhances antenna grounding reliability, but the illustrated configuration can add stiffness to the system, which may be visibly translated to the display.

FIG. 10E shows a similar configuration but with additional splits 1034 positioned between certain connection points 1030. The splits 1034 create discontinuities in a portion of the antenna ground ring 1014 while maintaining electrical paths through the connection points 1030 and continuous contact surface 1031. In this way, the antenna ground ring 1014 maintains its position relative to the overall assembly while adding flexibility to the antenna ground ring 1014. The added flexibility reduces the likelihood of image distortions imprinting on the display due to the antenna ground ring.

The arrangement demonstrates how the connection points 1030, continuous contact surface 1031, and splits 1034 work together to provide electrical connectivity while accommodating the geometric requirements of the device. The configuration allows for electrical contact between components while maintaining proper spacing and alignment within the assembly.

The figure shows a top view of an elongate conductor configured as an antenna ground ring 1014. The antenna ground ring 1014 follows a curved path that extends around a perimeter forming a continuous contact surface. Multiple connection points 1030 are positioned at intervals along the length of the antenna ground ring 1014.

As shown in FIG. 10F, the antenna ground ring 1014 includes splits 1034 at various locations along its length that create discontinuities in the internal portion of the ring structure. The splits 1034 separate different sections of the antenna ground ring 1014 and provide for added flexibility and compliance in the antenna ground ring 1014.

The connection points 1030 are distributed at specific locations around the curved path of the antenna ground ring 1014. The continuous contact surface 1031 maintains flatness of the overall antenna ground ring 1014 and electrical connectivity between segments while accommodating the curved geometry of the component. The splits 1034 divide the antenna ground ring 1014 into distinct sections while allowing the overall ring structure to maintain its configuration around the perimeter and provide continuous contact at the periphery. As noted above, the added flexibility and compliance added to the antenna ground ring 1014 via the splits 1034 can improve the overall image quality of an associated display disposed adjacent to the antenna ground ring in a stack-up.

Any of the features, components, and/or parts, including the arrangements and configurations thereof shown in FIGS. 10A-10F can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 10A-10F.

FIG. 11 shows a cross-sectional view of an electronic device 1100. In some examples, the electronic device 1100 can be a smart watch or other wearable electronic device. In some examples, the electronic device 1100 can include a metal frame 1101, a first insert 1103, and a second insert 1105. In some examples, the frame 1101 can carry operational components like batteries, vibrating mechanisms, processors, and cables, for example, in an internal volume at least partially defined by the housing. The frame 1103 can substantially define at least a portion of an exterior surface of the device 100. The frame 1013 can be manufactured from metal and can be additively manufactured with a variety of 3D printing techniques, including but not limited to powder bed fusion, direct energy deposition, electron beam melting, material jetting, and more. The frame 1101 can be manufactured from a variety of metals, including but not limited to aluminum alloys, steel, titanium, and more. The metal frame 1101 can provide a rigid and durable chassis for the electronic device 1100.

In some examples, the first insert 1103 and the second insert 1105 can be electronic components, user interface components, antennae, or plastic components. In some examples, the frame 1101 can be 3D printed, then the first insert 1103 and the second insert 1105 can be injection molded into the frame 1101. In other examples, the first insert 1103 and the second insert 1105 can be fixed into the frame 1101 with an adhesive. In yet other examples, the first insert 1103 and the second insert 1105 can be manufactured and secured to the frame 1101 with methods not described herein. In some examples, the first insert 1103 and the second insert 1105 can be thermoplastics, thermoset plastics, epoxy, or any other kind of plastic. In some examples, the first insert 1103 and the second insert 1105 can improve the structural rigidity, waterproof characteristics, or aesthetic qualities of the electronic device 1100.

In some examples, 3D printing the frame 1101 can facilitate internal features that are geometrically difficult or impossible with conventional machining methods. For example, the frame 1101 can include a retention feature 1107. As shown, retention feature 1107 is an undercut feature, and can be difficult or impossible using conventional methods like CNC machining. However, 3D printing the frame 1101 enables the frame 1101 to include undercut and other internal features. For example, the frame 1101 can also include hard-stop features that isolate internal components from one another, such as a hard-stop feature that prevents a batter assembly from contacting a display assembly in a high-g force event like the electronic device 1100 falling and hitting a surface or being bumped against an external object. Manufacturing the frame 1101 with additive manufacturing techniques like 3D printing also allows for the creation of passages and channels through the frame 1101 that are not straight, unlike subtractive drilling. These passages and channels that are not straight can further enable space-saving configurations of internal components, reducing the overall size of the electronic device 1100. Creation of difficult internal features through 3D printing can also reduce the amount of post-processing required after the frame 1101 is first manufactured, reducing wear on tooling, reducing labor costs, and increasing raw material utilization. 3D printing of the frame 1101 can also allow for internal features like retention feature 1107 to be smaller than the limits of subtractive manufacturing tooling. Retention feature 1107 can also improve the durability and strength of the electronic device 1100 by increasing the amount of force needed to dislodge the first insert 1103 from the frame 1101. In some examples, retention feature 1107 can form a hook, which geometrically locks the first insert 1103 in place and increases the force threshold that can dislodge the first insert 1103 from the frame 1101 by twenty percent, thirty-five percent, fifty percent, or more.

In some examples, a 3D printed frame 1101 can also include internal voids and/or closed internal cavities that are difficult or impossible to create with subtractive manufacturing methods. Internal cavities can help reduce weight of the electronic device 1100, allow for more room for electronic components withing the frame 1101, facilitate stronger reception of radio waves via an antenna, and reduce the amount of material needed to manufacture the frame 1101. Internal voids and cavities can also serve as walls for electronic components, eliminating the need for separate plastic housings that increase material usage and size. In some examples, 3D printing can be used to create internal lattice structures within the frame 1101 further reducing material usage while maintaining strength. In some examples, a minimum wall thickness of the frame 1101 can be determined based on rigidity and strength characteristics, and the frame 1101 can be 3D printed to maintain the smallest wall thickness possible utilizing internal voids and cavities. Reducing the amount of material used to manufacture the frame 11101 can also produce a lighter electronic device 1100, creating a desirable user experience. A lighter electronic device 1100 can also facilitate the use of a smaller, less expensive vibration mechanisms because the reduced mass can be vibrated with less force than a greater mass.

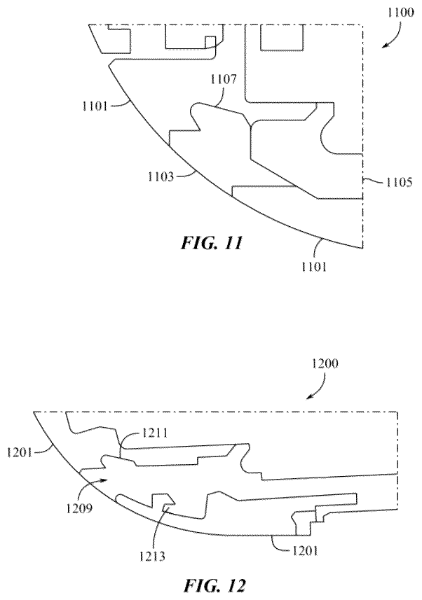

Any of the features, components, and/or parts, including the arrangements and configurations thereof shown in FIG. 11 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 11. More examples of 3D printed electronic device components, and their benefits are detailed below in reference to FIG. 12.

FIG. 12 shows a cross-sectional view of a portion of an electronic device 1200, including a frame 1201. In some examples, the electronic device 1200 can be a smart watch or other wearable electronic device. In some examples, the frame 1201 can include a channel 1209, and geometric features 1211, 1213. Like the frame 1101 described in reference to FIG. 11, the frame 1201 can also be manufactured from 3D printed metal. In some examples, the 3D printing process can leave the frame 1201 with a surface rougher than a machined surface, a sand-blasted surface, or an otherwise etched surface. Some surfaces of the frame 1201, such as the exterior surface, can be machined in post-processing to yield a desirable finish and feel. In some examples, the final visual characteristics and material properties of a 3D printed frame 1201 can be approximately identical to a cast or forged frame. In some examples, the channel 1209 can be manufactured through 3D printing to be smaller than drilled channels. In some examples, the geometric features 1211, 1213 and the channel 1209 can be filled with internal components. In some examples, the internal components can be plastic and can adhere to the surfaces of the channel 1209 and the geometric features 1211, 1213. The internal components can be injection molded into the frame 1201, fixed to the frame 1201 via an adhesive, or otherwise fixed to the frame 1201.

In some examples, the rough surfaces of the channel 1209 and the geometric features 1211, 1213 can improve the strength of the bond between the frame 1201 and any internal components affixed thereto. For example, the rough natural finish of the surfaces of the channel 1209 and the geometric features 1211, 1213 due to 3D printing can increase the surface area to which plastic components can be molded or fixed, thus improving the bond between the metal frame 1201 and any plastic components. A rough finish from 3D printing can eliminate the need for machining and etching processes which remove material and make the surface smooth, only to make the material rough again to increase the bond strength between metal and plastic components. Eliminating such post processing can reduce costs, reduce manufacturing time, reduce wear on tooling, and increase raw material utilization. All surfaces that are not post processed can include a rough finish left from 3D printing the frame 1201. For example, the inner surfaces of the channel 1209, the geometric features 1211, 1213, and other surfaces.

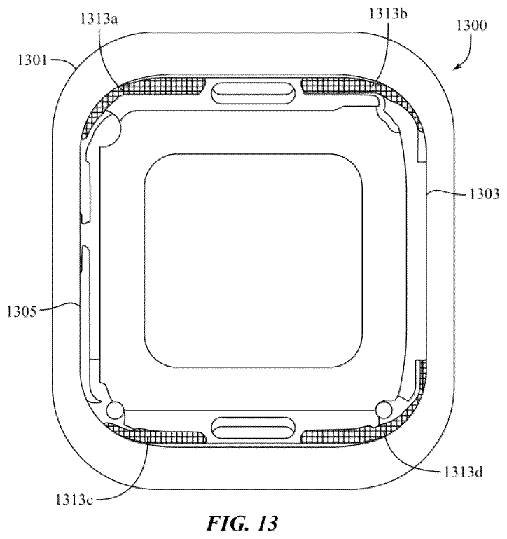

Any of the features, components, and/or parts, including the arrangements and configurations thereof shown in FIG. 12 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 12. More configurations and examples of 3D printed electronic device components, and their benefits are detailed below in reference to FIG. 13.

FIG. 13 shows a top cross-sectional view of an example of an electronic device 1300. In some examples, the electronic device 1300 can be a smart watch or other wearable electronic device. In some examples, the electronic device 1300 can include a frame 1301 and inserts 1303, 1305. In some examples, the frame 1301 can be manufactured from 3D printed metal, and the inserts 1303, 1305 can be manufactured from plastic. In some examples, the inserts 1303, 1305 can be injection molded into the frame 1301, fixed to the frame 1301 via an adhesive, or otherwise fixed to the frame 1301. The frame 1301 can also include texture regions 1313a-d produced during 3D printing of the frame 1301. In some examples, the texture regions 1313a-d can improve the bond between the inserts 1303, 1305 and the frame 1301. In some examples, the texture regions 1313a-d can differ from the natural rough finish of 3D printed metal. For example, the texture regions 1313a-d can be designed and include tubes, divots, raised portions, or other texture features that increase surface area of the bond between the inserts 1303, 1305 and the frame 1301.

In some examples, the tubes, divots, raised portions, or other textures of texture features of textured regions 1313a-d can be arranged randomly on a plane defined by the frame 1301, in a grid, or randomly on and above the plane in a foam-like structure. In some examples, the tubes can be round, hexagonal, or any other shape. In some examples, hexagonal tubes can enable a greater percentage of the texture regions 1313a-d compared to other texture features by reducing unused space between the individual features. In other examples, round tubes extending from the surface of the texture regions 1313a-d can be manufactured to be smaller than other texture features and therefore provide more surface area and a stronger bond between the frame 1301 and the inserts 1303, 1305. The tubes, divots, raised portions, or other textures of texture features of textured regions 1313a-d can be approximately 0.05 to 0.25 millimeters in diameter, 0.25 to 0.5 millimeters in diameter, or larger. In some examples, insert 1303, insert 1305, or both can bond to the texture regions 1313a-d with a stronger metal to plastic bond than an unaltered rough finish of the 3d printed frame 1301.

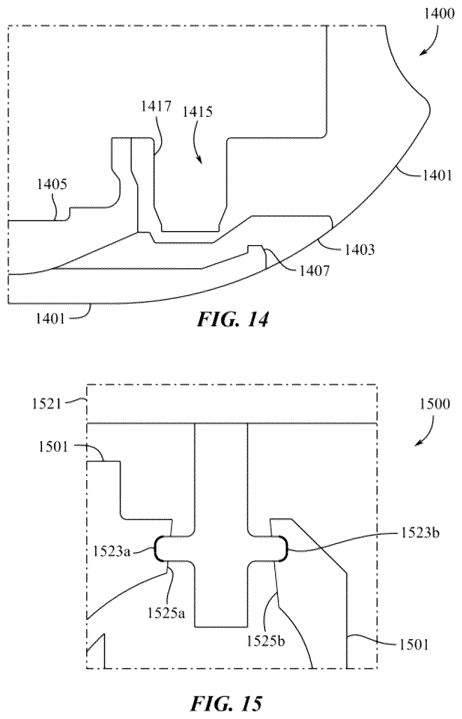

Any of the features, components, and/or parts, including the arrangements and configurations thereof shown in FIG. 13 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 13. Configurations and examples of 3D printed electronic device components including screw bosses are detailed below in reference to FIG. 14.

FIG. 14 shows a cross-sectional view of an example of a portion of an electronic device 1400. In some examples, the electronic device 1400 can be a smart watch or other wearable electronic device. In some examples, the electronic device 1400 can include a frame 1401 and inserts 1403, 1405. In some examples, the frame 1401 can be manufactured from 3D printed metal, and the inserts 1403, 1405 can be manufactured from plastic. In some examples, the inserts 1403, 1405 can be injection molded into the frame 1401, fixed to the frame 1401 via an adhesive, or otherwise fixed to the frame 1401. In some examples, the frame 1401 can also include a retention feature 1407, and a screw boss 1415. In some examples, the insert 1403 can occupy a channel in the frame 1401 that is not straight and would be difficult or impossible to manufacture with subtractive manufacturing techniques. The 3D printed frame 1401 also creates a rough surface to which the inserts 1403, 1405 can bond to, like the rough surface described in reference to FIG. 12.

As shown, 3D printing the frame 1401 allows for the screw boss 1415 to extend into the channel occupied by insert 1403, a geometry that would be difficult or impossible to manufacture by drilling or machining a solid part. The screw boss 1415 extending into the channel occupied by the insert 1403 can also create a stronger connection between a screw and the frame 1401 than a drilled hole occupied by the insert 1403 that would extend straight through the frame 1401 because a screw can extend further into the frame 1401 while engaging with metal than if the screw boss were shallower due to a straight channel. The screw boss 1415 extending into the channel occupied by the insert 1403 can also create a stronger connection between the insert 1403 and the frame 1401 than a drilled hole occupied by the insert 1403 that would extend straight through the frame 1401 because a screw of the same length would extend into the insert 1403, pushing the insert 1403 away from the frame 1403. Therefore, the complex geometries enabled by 3D printing the frame 1401 can improve the strength of the bond between the insert 1403 and the frame 1401, and the bond between fasteners and the frame 1401. In some examples, the wall 1417 can also be thinner than a subtractively manufactured screw boss wall, facilitating more room for electronic components like antennae, and therefore improving the wireless capabilities of the electronic device 1400.

Any of the features, components, and/or parts, including the arrangements and configurations thereof shown in FIG. 14 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 14. Configurations and examples of 3D printed electronic device components including both rough 3D printed surfaces and machined surfaces are described below in reference to FIG. 15.

FIG. 15 shows a cross-sectional portion of an example of an electronic device 1500. In some examples, the electronic device 1500 can be a smart watch or other wearable electronic device. In some examples, the electronic device 1500 can include a frame 1501 and a plastic component 1521. In some examples, the frame 1501 can be manufactured from 3D printed metal. In some examples, the plastic component 1521 can be fixed to the frame 1501. In some examples, the frame 1501 can include concave portions 1523a-b configured to receive the plastic component 1521. In some examples, the frame 1501 can also include cosmetic surfaces 1525a-b. In some examples, both the concave portions 1523a-b and the cosmetic surfaces 1525a-b can be manufactured with a rough surface finish from 3D printing. The cosmetic surfaces 1525a-b can then be machined to a different surface finish in post-processing to yield a desired aesthetic quality and user experience, while the concave portions 1523a-b can remain unchanged. In some examples, the unchanged concave portions 1523a-b with a rough surface finish can provide a strong bond between the plastic component 1521 and the frame 1501 without post processing, and the total amount of post processing, labor, and manufacturing time can be reduced by leaving the concave portions 1523a-b in the original 3D printed surface finish while allowing for easy post-processing of the cosmetic surfaces 1525a-b.

The 3D printing described above can be used to efficiently form near-final net shape components without excess post-processing such as machining and material waste. The 3D printing of metal parts, including the watch housing or other electronic device components and housings, can lead to unique geometry formations not possible with traditional molding, machining, and other manufacturing techniques. In addition, unique material properties and surface textures can be achieved on a single piece as part of a single 3D printing process.

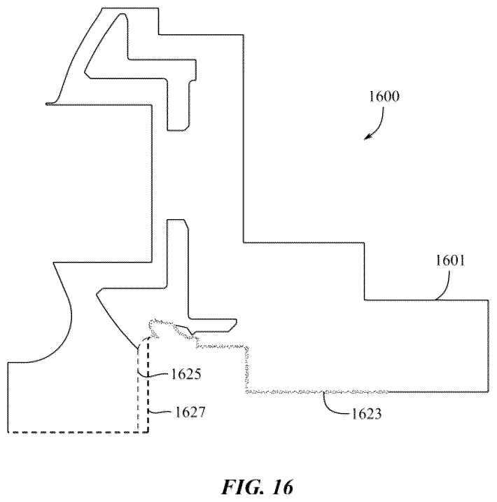

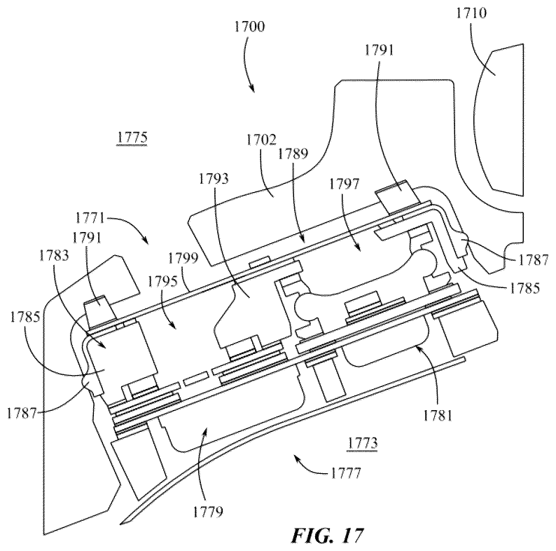

Any of the features, components, and/or parts, including the arrangements and configurations thereof shown in FIG. 15 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 15. More 3D printed frames for electronic devices are described below with emphasis on surface finish and post processing options in reference to FIG. 16.

FIG. 16 shows a cross-sectional portion of an example of an electronic device 1600. In some examples, the electronic device 1600 can be a smart watch or other wearable electronic device. In some examples, the electronic device 1600 can include a frame 1601. The frame 1601 can be substantially similar to the frame 1501 described in reference to FIG. 15. For example, the frame 1601 can be 3D printed and include a rough surface 1623. In the manufacturing process, the rough surface 1623 can be left as-is and remain unchanged in the final product. The frame 1601 can also include an etched surface 1627, which can start as a rough natural finish from 3D printing but be etched for a rougher finish and greater bonding strength to plastic components than the rough finish 1623. After etching the etched surface 1627, the frame 1601 can then be machined down to a cosmetic surface 1625. The frame 1601 can be designed with extra material between the etched surface to be removed to expose the cosmetic surface to allow the original finish to be etched, then the etched surface 1627 to be machined down to the cosmetic surface 1625 while maintaining the rest of the etched surface 1627 and preventing the marring of the machined surface 1625 due to etching. Designing the frame 1601 to include extra material to be machined away to expose a cosmetic surface 1625 of a desired finish can achieve a desired feel or aesthetic while minimizing the amount of material removed.

Any of the features, components, and/or parts, including the arrangements and configurations thereof shown in FIG. 16 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 16.

FIG. 17 shows a partial cross-sectional view of another example of a device 1700 including a housing 1702 defining an internal volume 1773. The housing 1702 can define an aperture 1771 through which the internal volume 1773 can be in fluid communication with an ambient environment 1775 external to the housing 1702. In some examples, the aperture 1771 shown in FIG. 17 can correspond to or be similar to the aperture 112 shown in FIG. 1B. In at least one example, the device can also include a button or crown 1710 similar to the button/crown 110 shown in FIG. 1B. In one example of the device 1700, a sensor assembly 1777 is disposed in the internal volume 1773.

In at least one example, the sensor assembly 1777 can include a first sensor, for example a microphone 1779, and a second sensor, for example an environmental sensor 1781, both of which can be coupled to or within a shared sensor housing 1783. The sensor housing 1783 can include an outer sidewall 1785 and a seal 1787 can be disposed and pressed between the sidewall 1785 and an internal surface of the device hosing 1702, as shown. The seal 1787 can define a shared front volume 1789 in fluid communication with the ambient environment 1775 through the aperture 1771. The microphone 1779 and the environmental sensor 1781 can also define the shared front volume 1789. The seal 1787 can be disposed between the sidewall 1785 of the sensor housing 1783 to isolate the internal volume 1773 from the shared front volume 1789 and thus the ambient environment 1775.

In the examples described herein, the environmental sensor 1781 can include a sensor detecting a state of the ambient environment 1775 external to the device housing 1702. In one example, the environmental sensor 1781 can include a pressure sensor. In one example, the environmental sensor 1781 can include a humidity sensor. In one example, the environmental sensor 1781 can include a temperature sensor. In one example, the environmental sensor 1781 can include a light sensor.

In one example, the seal 1787 can be a radial seal surrounding an outer perimeter of the sensor housing 1783 and the sensor assembly can further include a front seal 1791 disposed between the sensor housing 1783 and the device housing 1702. The front seal 1791 can also define and/or at least partially surround the front volume 1789. In at least one example, the sensor housing 1783 includes a dividing wall 1793 separating a first sub-volume 1795 of the shared front volume 1789 from a second sub-volume 1797 of the shared front volume 1789. The first sub-volume 1795 can be defined by and in fluid communication with the first sensor (e.g., microphone 1779) and the second sub-volume 1797 can be defined by and in fluid communication with the second sensor (e.g., environmental sensor 1781). The first and second sub-volumes 1795, 1797 can also be defined by the sidewall 1785 of the sensor housing 1783.

In at least one example, the sensor assembly 1777 can also include a plate 1799 coupled to the sensor housing 1783 to define the first and second sub-volumes 1795, 1797. The plate 1799 can be a perforated plate such that the first and second sub-volumes 1795, 1797 are in fluid communication with the shared front volume 1789 and thus in fluid communication with each other through the perforated plate 1799.

Any of the features, components, and/or parts, including the arrangements and configurations thereof shown in FIG. 17 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 17.

FIG. 18 shows a cross-sectional view of the sensor assembly 1777 isolated from the device 1700 and housing 1702 shown in FIG. 17. The sensor assembly 1777 shown in FIG. 18 can include the sensor housing 1785 having the dividing wall 1793 defining first and second sub-volumes 1795, 1797, respectively. The sensor assembly 1777 also includes the perforated plate 1799 coupled to the sensor housing 1783, including coupled to the sidewall 1785 and the dividing wall 1793, to define the sub-volumes 1795, 1797. In at least one example, the perforated plate 1799 can be coupled directly to the dividing wall 1793 and sidewall 1785 via adhesives, welding, epoxy, and the like. In at least one example, the perforated plate 1799 can be disposed between the radial seal 1787 and the front seal 1791.

The perforated plate 1799 can be coupled to the housing 1783 and extend across the inner dividing wall 1793, with the first sub-volume 1795 in fluid communication with the second sub-volume 1797 via one or more perforations extending through the perforated plate 1799. In at least one example, the perforated plate 1799 is disposed between the first and second sensors 1779, 1781 and the front volume 1789.

Any of the features, components, and/or parts, including the arrangements and configurations thereof shown in FIG. 18 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 18.

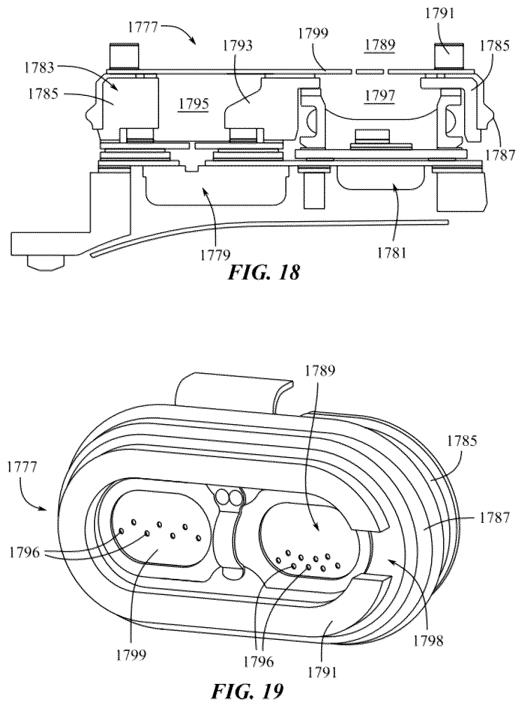

FIG. 19 shows a perspective view of the sensor assembly 1777, including the front seal 1791, housing 1785, radial seal 1785, and perforated plate 1799. The front seal 1791 and the perforated plate 1799 can define the front volume 1789 between the sensor assembly 1777 and the device housing 1702, shown in FIG. 17. In at least one example, the perforated plate 1799 is disposed between the sensor housing 1785 and the front seal 1791. As shown in FIG. 19, one example of the front seal 1798 can be a C-shape defining a gap 1798 between terminal ends of the front seal 1791. In this way, the front seal 1791 can extend partially around the first and second sub-volumes 1795, 1797 and the perforated plate 1799. FIG. 19 also shows perforations 1796, which can vary in number, shape, and size in different examples.

The gap 1798 defined by the front seal 1791 can form a passageway for water, other liquid, or other debris that enters from the ambient environment 1775 into the front volume 1789 through the aperture 1771 in the device housing 1702 to exit through one or more other ports or passageways defined through the housing 1702. In this way, drying time and debris egress can be facilitated by the gap 1798.

Any of the features, components, and/or parts, including the arrangements and configurations thereof shown in FIG. 19 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 19.

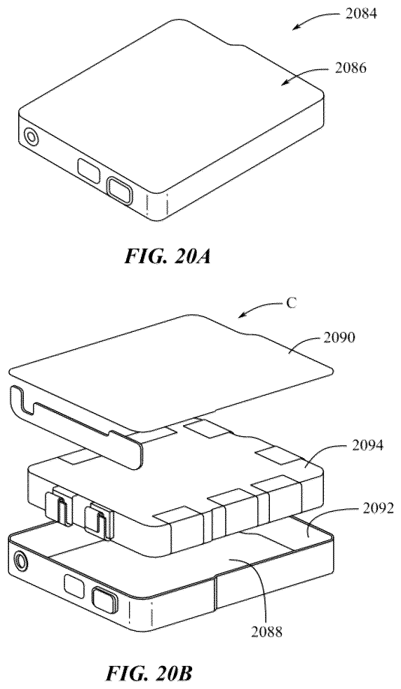

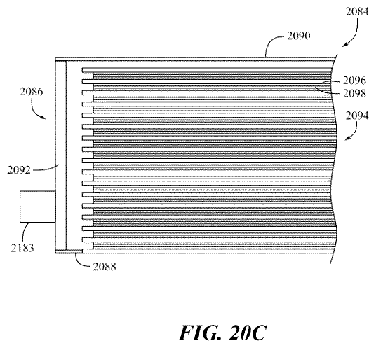

FIG. 20A shows a top view, an exploded view, and a cross-sectional view of a battery cell 2084 of a wearable electronic device. The battery cell 2084 can include a housing 2086 defining an internal volume. The housing 2086 can include a first housing plate 2088, a second housing plate 2090, and a housing sidewall 2092. A battery assembly 2094 can be disposed in the internal volume defined by the housing 2086. In an example, the internal volume also can include a component. The component can be an electrical component, a mechanical component, or a combination thereof.

The first housing plate 2088 can be positioned at a bottom of the battery assembly 2094. The first housing plate 2088 can have a rectangular shape. The first housing plate 2088 can include a cutout C in the rectangular shape. The first housing plate 2088 can have a first thickness. The first thickness can be about 25 microns to about 75 microns. The first thickness can be about 40 microns to about 60 microns. The first thickness can be about 50 microns.

The second housing plate 2090 can be positioned at a top of the battery assembly 2094. The second housing plate 2090 can be substantially parallel to the first housing plate 2088. The second housing plate 2090 can have a size and shape similar to the first housing plate 2088. For example, the first housing plate 2090 and the second housing plate 2088 can be the same shape with the same cutout C corresponding in shape and size. The similar shaped plates 2088, 2090 can be similar enough in size that a housing sidewall 2092 (discussed in more detail below), can be substantially perpendicularly oriented (90-degree orientation plus-or-minus about 5-degrees) relative to both plates 2090, 2088 and coupled to each plate 2088, 2090 positioned parallel to one another. The second housing plate 2090 can have a rectangular shape. The second housing plate 2090 can include a cutout C in the rectangular shape. The second housing plate 2090 can have a second thickness. The second thickness can be substantially similar to the first housing plate 2088. The second thickness can be about 25 microns to about 75 microns. The second thickness can be about 40 microns to about 60 microns. The second thickness can be about 50 microns.

The housing sidewall 2092 can be substantially perpendicular to the first housing plate 2088 and the second housing plate 2090. The housing sidewall 2092 can surround the battery assembly 2094. The housing sidewall 2092 can be positioned along a perimeter of the first housing plate 2088 and the second housing plate 2090. The housing sidewall 2092 can have a third thickness. The third thickness can be greater than the first thickness and/or the second thickness. The third thickness can be twice as thick as the first thickness and/or the second thickness. The third thickness can be about 100 microns to about 300 microns. The third thickness can be about 175 microns to about 225 microns. The housing sidewall 2092 can be welded to the first housing plate 2088. The housing sidewall 2092 can be welded to the second housing plate 2090. The housing sidewall 2092 can be welded to a housing of the wearable electronic device. The housing sidewall 2092 can be welded directly to the housing of the wearable electronic device. The housing sidewall 2092 can be welded to the housing of the wearable electric device via a bracket. The housing sidewall 2092 can be welded directly to the bracket. The component can be welded to the housing sidewall 2092. The component welded to the housing sidewall 2092 can be an electrical component, a structural component, or a combination thereof. The component can be welded to the housing sidewall 2092 in the internal volume of the battery cell 2084. The component can secure the battery assembly 2094 to the housing sidewall 2092.

The battery assembly 2094 can include an anode layer 2096 and a cathode layer 2098. The battery assembly 2094 can include many anode layers 2096 and many cathode layers 2098. The anode layer 2096 and the cathode layers 2098 can be cut in any shape. The battery assembly 2094 can be shaped to maximize space within the internal volume, thus increasing performance.

Any of the features, components, and/or parts, including the arrangements and configurations thereof shown in FIGS. 20A-20C can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 20A-20C.

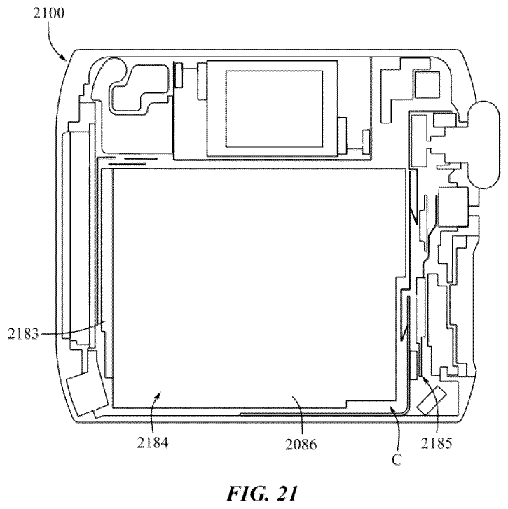

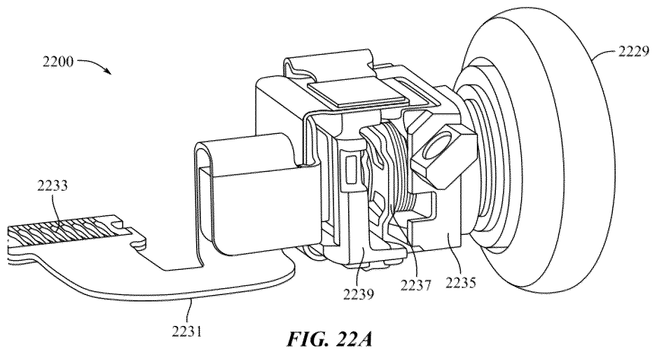

FIG. 21 shows a top view of a battery cell 2184 within a wearable electronic device 2100. The battery cell 2184 can include a housing 2086. The housing 2086 can include one or more cutouts C. The cutout C can allow the battery cell 2184 to nest within the wearable electronic device 2100. For example, the cutout C can allow the battery cell 2184 to nest within other components of the electronic device 2100. Thus, the battery cell 2184 can optimize space within the wearable electronic device 2100, while also maximizing battery volume, and therefore, battery energy.

In one example, components 2183, such as the various brackets, electronic components, and other components noted above, can be welded directly to the battery cell 2184, including directly to the sidewall 2092 of the battery housing 2086. In this way, the sidewall 2092 of the battery housing 2086 can serve as a structural component supporting brackets or other components welded directly thereto, as shown in FIG. 21 with the component 2183 welded directly to the sidewall 2092 of the battery housing 2186.

In addition, the cutout C can accommodate adjacent components, such as components 2185, which can be positioned within the space available from cutout C to save space within the device 2100. The components 2185 nested or positioned within the area provide by the cutout C can be electrical wires, cables, PCBs, structural brackets, computing components, or any other component within the device 2100 or combinations thereof.

Any of the features, components, and/or parts, including the arrangements and configurations thereof shown in FIG. 21 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 21.

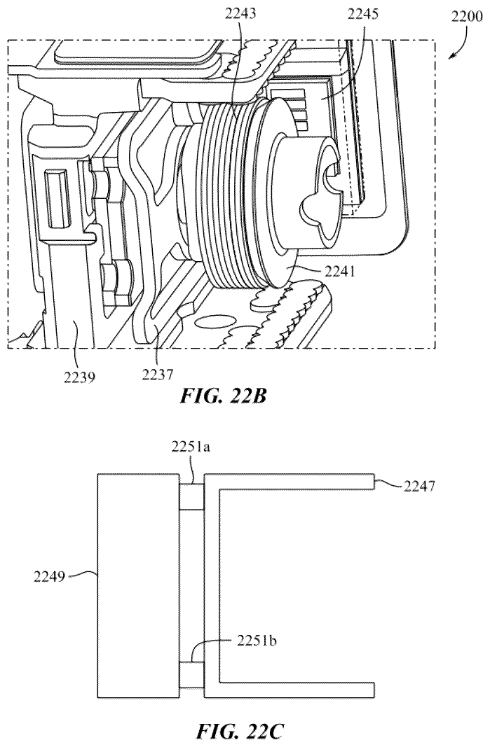

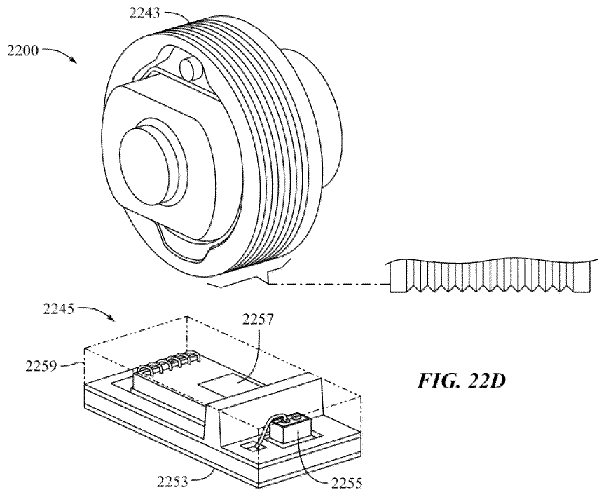

FIGS. 22A-22D show various views of an example of a crown assembly 2200 for a wearable electronic device. In some examples, the electronic device can be a smart watch or other wearable electronic device. FIG. 22A shows the crown assembly 2200, which can include a crown 2229, a flexible electrical connector 2231, an electrical interface 2233, a bucket 2235 configured to receive the crown 2229, a switch 2237, and a chassis 2239. In some examples, the crown 2229 is configured to be driven by a user to spin about an axis and translate along the same axis. In some examples, the crown 2229 can be a user input mechanism, whereby a user can control one or more functionalities of an electronic device. For example, a user can twist the crown to set timers, scroll through menu options, scroll through web pages, or other functions. A user can also push the crown to select various options displayed to the user. In some examples, the crown 2229 can be coupled to the switch 2237, and the switch 2237 can be bi-stable to produce a clicking sensation to a user when the crown is pressed.

In some examples, the bucket 2235 can be configured to receive the crown 2229 and can fluidly seal the rest of the crown assembly 2200 from the crown 2229. In some examples, the crown assembly 2200 can include various seals and/or gaskets to prevent foreign contaminants including dust from entering the crown assembly 2200. In some examples, the bucket 2235, the chassis 2239 or both can secure the crown assembly 2200 to other components of an electronic device, like a frame or housing. For example, the bucket 2235 can include an angled screw point configured to receive a fastener and secure the crown assembly to a larger electronic device. In some examples, the flexible electrical connector 2231 can electrically transmit information relating to the position of the crown 2229 to a processor. In some examples, the electrical interface 2233 can be a zero-insertion force electrical connector, which can be smaller than alternative connectors and reduce the overall size of the crown assembly 2200.

FIG. 22B shows a close-up of the crown assembly 2200, including the switch 2237, the chassis 2239, a crown shaft 2241, a reflector 2243, and a sensor 2245. In some examples, the crown shaft 2241 can be secured to the crown 2229 or integrally formed with the crown 2229. In some examples, the reflector 2243 can be secured to the crown shaft 2241. In some examples, the sensor 2245 can be secured to the chassis 2239. The sensor 2245 can shine a light at the reflector 2243, and the reflector 2243 can reflect the light back to the sensor 2245 to determine the rotational position of the crown shaft 2241 and therefore the crown 2229. The crown shaft 2241 can extend from the crown 2229 and contact the switch 2237. The switch 2237 can be configured to receive the crown shaft 2241 and translate in response to a translation of the crown shaft 2241. In some examples, the crown shaft 2241 and the switch 2237 are also electrically coupled to one another and can transmit an electrical signal from the crown shaft 2241 through the switch 2237. In some examples, the switch can be made from stainless steel and finished with protective coating to prevent wear from contact with the crown shaft 2241 and increase the longevity of the crown assembly 2200. In some examples, the protective coating can be a diamond-like carbon coating. The thickness of the coating can be determined by optimizing for strength of electrical connection while maintaining durability.

In some examples, the switch 2237 can also be greased with a conductive grease to improve the electrical connection between the crown shaft 2241 and the switch 2237. In some examples, the switch 2237 can be configured so an angle of the surface contacting the crown shaft remains approximately level as the switch 2237 translates. A relatively level surface of the switch 2237 can prevent the conductive grease from being pushed away from the connection with the crown shaft 2241 and therefore increase the longevity of the crown assembly 2200. In some examples, the crown shaft 2241 can further include a separate connection arm extending from the crown shaft 2241 and configured to always be in electrical communication with the switch 2237. For example, the connection arm can be a spring-loaded conductive member configured to always contact the switch 2237 regardless of distance or angle. In some examples, the switch geometry is designed to maintain a level surface contacting the crown shaft 2241 while achieving a desired force profile in translation. In some examples, the force profile can be configured to produce a satisfying click feeling when the crown 2229 is pressed. In some examples, the switch 2237 can have various relief cuts, part thicknesses, and bending lengths to achieve the desired force profile through a click.

FIG. 22C shows a cross-sectional view of a portion of the crown assembly 2200. The crown assembly can include a bracket 2247 coupled to the chassis 2239, the switch 2237, or other components of the crown assembly 2200. In some examples, the bracket can be manufactured from metal or plastic. The crown assembly 2200 can also include a chiplet 2249 secured to the bracket 2247 via adhesive members 2251a-b. A chiplet can refer to modular integrated circuit that can be combined with other electronic components. In some examples, the adhesive members 2251a-b can be pressed sensitive adhesives. In some examples, the bracket 2247 can be configured to deflect in response to an external force such as a drop event, causing the bracket 2247 to bow with an apex extending toward the chiplet 2249. The deflection of the bracket 2247 can be tuned by the material properties and/or the geometry of the bracket 2247. For example, various holes or slits can be included in the bracket 2247 to change the bending properties. Additionally, holes or slits in the bracket 2247 can be used to affix the bracket 2247 to other crown assembly components. In some examples, the bracket 2247 can also include chamfered edges to improve adhesion to other components of the crown assembly 2200.

In some examples, the adhesive members 2251a-b can have a thickness designed to create space between the chiplet 2249 and the bracket 2247 so that the bracket 2247 does not damage the chiplet 2249 as the bracket 2247 deflects and bows. In some examples, the gap can be configured such that the bracket 2247 touches the chiplet 2249 as the bracket 2247 bows but does not press on the chiplet 2249 with enough force to damage the chiplet 2249. In other examples, the gap can be configured such that the bracket 2247 does not touch the chiplet 2249 as the bracket 2247 bows. In some examples, the width of the adhesive members 2251a-b can be configured to create a gap between the bracket 2247 and the chiplet 2249 wide enough to protect the chiplet 2249 from damage while minimizing the width of the gap and therefore the overall size of the crown assembly 2200. In some examples, the adhesive members 2251a-b can be approximately 50 to 100 microns thick. As shown in FIG. 22C, the adhesive members 2251a-b can be disposed as far apart along the surface of the chiplet 2249 as possible to increase the length of the portion of the bracket 2247 that bends, reducing the translation of the chiplet 2249 and protecting the chiplet 2249 from abutting other components and reduce the size of the crown assembly 2200.

FIG. 22D shows a portion of the crown assembly 2200, including the reflector 2243 and the sensor 2245. The sensor 2245 can include a base 2253, a light source 2255, a light sensor 2257, and a transparent portion 2259. The light source 2255 can be a light-emitting diode. In some examples, the transparent portion 2259 can be injection molded over the base 2253, the light source 2255, and the light sensor 2257. The process of injection molding a plastic part over other parts can be referred to as overmolding. Overmolding of the transparent layer 2259 onto the base 2253, the light source 2255, and the light sensor 2257 can decrease the size of the sensor 2245 and therefore the size of the crown assembly 2200. A smaller sensor 2245 can also reduce material costs, reduce the carbon footprint of manufacturing, and reduce manufacturing times.

In some examples, the reflector 2243 can be retro-reflective. In some examples, a retro-reflective reflector 2243 can increase the amount light emitted from the light source 2255 that is reflected to the light sensor 2257 compared to traditional reflective surfaces. The reflector 2243 can be a plastic or metal part coated with a retro-reflective material. In some examples, the increased amount of light directed toward the light sensor 2257 due to a retro-reflective reflector 2243 can increase the sensitivity of the sensor 2245. In some examples, light-blocking features like opaques tapes, gaskets, seals, and other measures can help prevent light outside the crown assembly 2200 from entering the light sensor 2257 and effecting the measured position of the crown 2229.

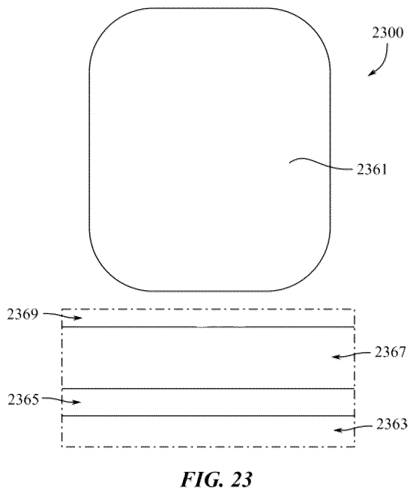

Any of the features, components, and/or parts, including the arrangements and configurations thereof shown in FIGS. 22A-22D can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIGS. 22A-22D . Electronic device components relating to transparent covers and durable coatings are described below in reference to FIG. 23.

FIG. 23 shows an example transparent cover 2300 for an electronic device. In some examples, the electronic device can be a smart watch or other wearable electronic device including a screen, and the transparent cover 2300 can be configured to cover and protect the screen. In some examples, the transparent cover 2300 can improve the durability and appearance of an electronic device. In some examples, the transparent cover 2300 can include curved edges, curved corners, and define both flat and curved surfaces. In some examples, the transparent cover 2300 can include a coating. The coating can include multiple layers. For example, the coating can include a glass layer 2363, ceramic layers 2365, 2367, and an anti-smudging layer 2369 to reduce fingerprint marks. In some examples, ceramic layer 2365 can be silicone dioxide. In some examples, ceramic layer 2367 can be silicone oxynitride. In some examples, the ceramic layers 2365 can be approximately 200 nanometers thick. In some examples, the ceramic layer 2367 can be approximately 2 microns thick. Additionally, in some examples, the anti-smudging layer 2369 can be approximately 10 microns thick and can be an oleophobic layer that lacks a strong affinity for oils, or any material or layer that repels oils and prevents fingerprints on the surface.

In some examples, the glass layer 2363 can transition from glass to the ceramic layer 2365, and further to the ceramic layer 2367 can via a gradient of materials. A gradient of materials can include a transition portion where different materials mix with varying levels of concentration throughout the transition portion and generally trend from a higher of concentration of one material to a higher concentration of another material. The gradient can help improve bonding between the layers, reduce materials costs, and facilitate manufacturing. In some examples, the coating can increase the scratch resistance of the transparent cover 2300. The coating can also prevent cracking, and prevent damage from drop events, and increase product life. In some examples, the coating can increase the scratch resistance of the transparent cover 2300 by approximately one hundred percent. Additionally, the glass layer 2363 and ceramic layers 2365, 2367 can also prevent cracking due to impact from foreign objects and prevent cracking from bending or other applied stresses.

In some examples, the transparent cover 2300 can include a number of foreign particles under a specified threshold. In some examples, the manufacturing process can include the application of multiple layers while preventing particulates or other contaminants from entering the transparent cover 2300. In some examples, the manufacturing process can be configured to prevent particulates of larger than approximately 0.005 millimeters from entering the transparent cover layer. In other examples, the manufacturing process can reduce particles of a certain size or shape that are especially noticeable to the human eye, such as oval shaped particles or scattered particles. In some examples, the transparent cover 2300 can shift the wavelengths of light passing through the transparent cover 2300. In some examples, the light passing through the transparent cover 2300 can be emitted from a screen. In some examples, the degree of light distortion due to the transparent cover 2300 can be configured to produce a desired hue, warmth, or brightness. In other examples, the change in wavelength of light passing through the transparent cover 2300 can be corrected via an additional film, lens, or cover.

Any of the features, components, and/or parts, including the arrangements and configurations thereof shown in FIG. 23 can be included, either alone or in any combination, in any of the other examples of devices, features, components, and parts shown in the other figures. Likewise, any of the features, components, and/or parts, including the arrangements and configurations thereof shown in the other figures can be included, either alone or in any combination, in the example of the devices, features, components, and parts shown in FIG. 23.

To the extent applicable to the present technology, gathering and use of data available from various sources can be used to improve the delivery to users of invitational content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, TWITTER® ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An electronic device, comprising:
a display assembly;
a sidewall defining an internal volume including four corners;
an elongate conductor extending between the display assembly and the sidewall, the elongate conductor including a plurality of contact points spaced around a perimeter of the display assembly, each contact point of the plurality of contact points being welded to the housing;
a printed circuit board (PCB) disposed in the internal volume, the PCB including a continuous conductive element that extends around the printed circuit board; and
an electrical connector contacting the PCB, the electrical connector contacting the elongate conductor and forming an electrical contact between the sidewall and the PCB.

2. The electronic device of claim 1, wherein the sidewall comprises:
an upper portion;
a lower portion; and
a non-conductive split disposed between the upper portion and the lower portion.

3. The electronic device of claim 2, further comprising an antenna assembly including a resonator, the resonator comprising the upper portion.

4. The electronic device of claim 2, wherein the PCB comprises an antenna ground contact and the electrical connector contacts the antenna ground contact.

5. The electronic device of claim 4, wherein the electrical connector is welded to the elongate conductor.

6. The electronic device of claim 1, wherein the plurality of contact points comprises twelve discrete contact points.

7. The electronic device of claim 1, wherein:
the PCB defines four corners; and
the elongate conductor and the continuous conductive element are electrically coupled at each of the four corners.

8. The electronic device of claim 1, wherein the elongate conductor further comprises:
an elongate body;
wherein each contact point is independently rotated from the elongate body.

9. An electronic device, comprising:
a conductive housing sidewall defining an internal volume;
a printed circuit board (PCB) disposed in the internal volume;
an electrical connector contacting the PCB; and
an elongate conductive member disposed between the housing sidewall and the electrical connector, the elongate conductive member contacting the electrical connector and welded to housing sidewall;
wherein:
the elongate conductive member defines a plurality of contact points;
each contact point of the plurality of contact points is decoupled and rotated from the elongate body and welded to the housing.

10. The electronic device of claim 9, wherein the elongate conductive member contacts the electrical connector at a first location along a length of the elongate conductive member.

11. The electronic device of claim 10, wherein:
the elongate conductive member contacts the electrical connector at a third location along the length of the elongate conductive member; and
the elongate conductive member is welded to the housing sidewall at a fourth location along the length of the elongate conductive member.

12. The electronic device of claim 9, wherein the conductive housing sidewall comprises a resonating element of an antenna.

13. The electronic device of claim 12, wherein the elongate conductive member is welded to the resonating element.

14. The electronic device of claim 9, wherein the electrical connector comprises a continuous component disposed adjacent the PCB.

15. The electronic device of claim 9, wherein the electrical connector extends through an insulating material between the PCB and the conductive housing sidewall.

16. The electronic device of claim 15, wherein:
the conductive housing defines an opening;
the electronic device further comprises a display disposed in the opening; and
the elongate conductive member is disposed between the display and the conductive housing.

17. A wearable electronic device, comprising:
a housing sidewall including a lower portion and an electrically conductive upper portion separated from the lower portion by a non-conductive material, the housing sidewall defining an opening;
a display component disposed in the opening;
an antenna ground contact;
a display printed circuit board (display PCB) electrically coupled to the antenna ground contact;
a wave ring disposed between the electrically conductive upper portion and the display component, the wave ring welded to the electrically conductive upper portion at a plurality of discrete contact points disposed along a length of the wave ring;
a connector forming an electrical pathway between the wave ring and the antenna ground contact;
a sensor printed circuit board (sensor PCB) disposed within the housing sidewall; and
a monopole antenna disposed on the sensor PCB that unifies the electrically conductive upper portion with the sensor PCB.

18. The electronic device of claim 17, wherein the upper conductive portion and the sensor PCB each comprise a resonating element of the monopole configuration.

19. The electronic device of claim 17, wherein the monopole antenna includes a coax connection to the sensor PCB.

20. The electronic device of claim 17, wherein the monopole antenna further comprises a four-layer flex.
